SEQUENCE LISTING (69 sequences)

SEQ ID NO: 1 - RNA, 10 nt, Artificial Sequence
  Synthetic
cggaaagacc

SEQ ID NO: 2 - DNA, 11 bp, Artificial Sequence
  Synthetic
cgtctagagca

SEQ ID NO: 3 - protein, 131 aa, Artificial Sequence
  Synthetic
METDTLLLWVLLLWVPSTGDIVLTQSPASLAVSLGQRATISCRASQSVSTSSYSYMHWYQQKPGQPPKLLIKYASNLESGVPARFSGSGSGTDFTLNIHPVEEEDTATYYCQHSWEIPYTFGGGTKLEIKR

SEQ ID NO: 4 - DNA, 393 bp, Artificial Sequence
  Synthetic
atggagacagacacactcctgctatgggtgctgctgctctgggttccatccactggtgacattgtgctaacacagtctcctgcttccttagctgtatctctggggcagagggccaccatctcatgcagggccagccaaagtgtcagtacatctagctatagttatatgcactggtatcaacagaaaccaggacagccacccaaactcctcatcaagtatgcttccaacctagaatctggggtccctgccaggttcagtggcagtgggtctgggacagacttcaccctcaacatccatcctgtggaggaggaggatactgcaacatattactgtcagcacagttgggagattccgtacacgttcggaggggggaccaagctggaaataaaacgg

SEQ ID NO: 5 - protein, 18 aa, Artificial Sequence
  Synthetic
GSTSGSGKPGSGEGSTKG

SEQ ID NO: 6 - DNA, 54 bp, Artificial Sequence
  Synthetic
ggatcaacttcgggcagtggtaagcctggtagtggtgagggtagtaccaagggc

SEQ ID NO: 7 - protein, 117 aa, Artificial Sequence
  Synthetic
QIQLVQSGPELKKPGETVKISCKASGYTFTDYSMHWVKQAPGKGLKWMGWINTETGEPTYADDFKGRFAFSLETSASTAYLQINNLKNEDTATYFCARGKYGAFAYWGQGTLVTVSA

SEQ ID NO: 8 - DNA, 351 bp, Artificial Sequence
  Synthetic
cagatccagttggtgcagtctggacctgagctgaagaagcctggagagacagtcaaaatctcctgcaaggcttctggttataccttcacagactattcaatgcactgggtgaagcaggctccaggaaagggtttaaagtggatgggctggataaacactgagactggtgagccaacgtatgcagatgacttcaagggacggtttgccttctctttggaaacctctgccagcactgcctatttgcagatcaacaacctcaaaaatgaggacacggctacatatttctgtgctagaggaaagtatggggcctttgcttactggggccaagggactctggtcactgtctcagca

SEQ ID NO: 9 - protein, 48 aa, Artificial Sequence
  Synthetic
TTTPAPRPPTPAPTIASQPLSLRPEACRPAAGGAVHTRGLDFACDISS

SEQ ID NO: 10 - DNA, 144 bp, Artificial Sequence
  Synthetic
accacaacgccagctccccgcccaccaacgcctgcgccaactattgcctcacagcctttgagtctccggccagaagcatgtcgccccgctgccggtggagcagtccatacaagaggccttgacttcgcgtgcgatatctcgagc

SEQ ID NO: 11 - protein, 66 aa, Artificial Sequence
  Synthetic
FWVLVVVGGVLACYSLLVTVAFIIFWVRSKRSRLLHSDYMNMTPRRPGPTRKHYQPYAPPRDFAAY

SEQ ID NO: 12 - DNA, 198 bp, Artificial Sequence
  Synthetic
ttctgggtgttggtcgttgtgggtggtgtcctggcgtgttattcactgttggttactgtggcttttataattttctgggtgaggagtaagaggagcaggctcctgcacagtgactacatgaacatgactccccgccgcccagggccaacccgcaagcattaccagccctatgccccaccacgcgacttcgcagcctat

SEQ ID NO: 13 - protein, 40 aa, Artificial Sequence
  Synthetic
RSQVRKAAITSYEKSDGVYTGLSTRNQETYETLKHEKPPQ

SEQ ID NO: 14 - DNA, 123 bp, Artificial Sequence
  Synthetic
aggtctcaagttagaaaagcagctataacatcttatgagaaatctgatggagtatatacagggctcagcacgcgaaatcaggagacctatgaaactctgaagcatgagaagcccccgcagtag

SEQ ID NO: 15 - DNA, 17 bp, Artificial Sequence
  Synthetic
ggcggccgcgaattcgc

SEQ ID NO: 16 - protein, 131 aa, Artificial Sequence
  Synthetic
MRFSAQLLGLLVLWIPSTADIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPWTFGGGTKLEIK

SEQ ID NO: 17 - DNA, 393 bp, Artificial Sequence
  Synthetic
atgaggttctctgctcagcttctggggctgcttgtgctctggataccttccacagcagatattgtgatgacgcaggctgcattctccaatccagtcactcttggaacatcagcttccatctcatgcaggtctagtaagagtctcctacatagtaatggcatcacttatttgtattggtatctacagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctcgaacttccgtggacgttcggtggaggcaccaagctggaaatcaaa

SEQ ID NO: 18 - protein, 121 aa, Artificial Sequence
  Synthetic
ETVRHRGPCAPDIEVPVRPSNSCTVISGTVLSSQGVHLKIEDVLGIISGDGEPTLHRCTVLCCVTISFVSNKTQPLKCLSWRLADPAHVVISEGEPRSCTGESQRTPRLYQASSRLHQLHL

SEQ ID NO: 19 - DNA, 362 bp, Artificial Sequence
  Synthetic
gaggtgaagctggtggagtctggaggaggcttggtacagcctgggggttctctgagactctcctgtgcaacttctgggttcaccttcactgattactacatgagctgggtccgccagcctccaggaaaggcacttgagtggttgggttttattagaaacaaagctaatggttacacaacagagtacagtgcatctgtgaagggtcggttcaccatctccagagataattcccaaaacatcctctatcttcaaatgaacaccctgagagctgaggacagtgccacttattactgtgcaagagttactgggacgcactggtacttcgatgtctggggcgcagggaccacggtgtctcaccgtct

SEQ ID NO: 20 - protein, 588 aa, Artificial Sequence
  Synthetic
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRT

SEQ ID NO: 21 - DNA, 1764 bp, Artificial Sequence
  Synthetic
atgccacatactttgtggatggtgtgggtcttgggggtcatcatcagcctctccaaggaagaatcctccaatcaggcttctctgtcttgtgaccgcaatggtatctgcaagggcagctcaggatctttaaactccattccctcagggctcacagaagctgtaaaaagccttgacctgtccaacaacaggatcacctacattagcaacagtgacctacagaggtgtgtgaacctccaggctctggtgctgacatccaatggaattaacacaatagaggaagattctttttcttccctgggcagtcttgaacatttagacttatcctataattacttatctaatttatcgtcttcctggttcaagcccctttcttctttaacattcttaaacttactgggaaatccttacaaaaccctaggggaaacatctcttttttctcatctcacaaaattgcaaatcctgagagtgggaaatatggacaccttcactaagattcaaagaaaagattttgctggacttaccttccttgaggaacttgagattgatgcttcagatctacagagctatgagccaaaaagtttgaagtcaattcagaatgtaagtcatctgatccttcatatgaagcagcatattttactgctggagatttttgtagatgttacaagttccgtggaatgtttggaactgcgagatactgatttggacactttccatttttcagaactatccactggtgaaacaaattcattgattaaaaagtttacatttagaaatgtgaaaatcaccgatgaaagtttgtttcaggttatgaaacttttgaatcagatttctggattgttagaattagagtttgatgactgtacccttaatggagttggtaattttagagcatctgataatgacagagttatagatccaggtaaagtggaaacgttaacaatccggaggctgcatattccaaggttttacttattttatgatctgagcactttatattcacttacagaaagagttaaaagaatcacagtagaaaacagtaaagtttttctggttccttgtttactttcacaacatttaaaatcattagaatacttggatctcagtgaaaatttgatggttgaagaatacttgaaaaattcagcctgtgaggatgcctggccctctctacaaactttaattttaaggcaaaatcatttggcatcattggaaaaaaccggagagactttgctcactctgaaaaacttgactaacattgatatcagtaagaatagttttcattctatgcctgaaacttgtcagtggccagaaaagatgaaatatttgaacttatccagcacacgaatacacagtgtaacaggctgcattcccaagacactggaaattttagatgttagcaacaacaatctcaatttattttctttgaatttgccgcaactcaaagaactttatatttccagaaataagttgatgactctaccagatgcctccctcttacccatgttgctagtattgaaaatcagtaggaatgcaataactacgttttctaaggagcaacttgactcatttcacacactgaagactttggaagctggtggcaataacttcatttgctcctgtgaattcctctccttcactcaggagcagcaagcactggccaaagtcttgattgattggccagcaaattacctgtgtgactctccatcccatgtgcgtggccagcaggttcaggatgtccgcctctcggtgtcggaatgtcacaggaca

SEQ ID NO: 22 - protein, 784 aa, Homo sapiens
  VARIANT (681)..(681): PRO to HIS substitution
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 23 - DNA, 2355 bp, Homo sapiens
atgccacatactttgtggatggtgtgggtcttgggggtcatcatcagcctctccaaggaagaatcctccaatcaggcttctctgtcttgtgaccgcaatggtatctgcaagggcagctcaggatctttaaactccattccctcagggctcacagaagctgtaaaaagccttgacctgtccaacaacaggatcacctacattagcaacagtgacctacagaggtgtgtgaacctccaggctctggtgctgacatccaatggaattaacacaatagaggaagattctttttcttccctgggcagtcttgaacatttagacttatcctataattacttatctaatttatcgtcttcctggttcaagcccctttcttctttaacattcttaaacttactgggaaatccttacaaaaccctaggggaaacatctcttttttctcatctcacaaaattgcaaatcctgagagtgggaaatatggacaccttcactaagattcaaagaaaagattttgctggacttaccttccttgaggaacttgagattgatgcttcagatctacagagctatgagccaaaaagtttgaagtcaattcagaatgtaagtcatctgatccttcatatgaagcagcatattttactgctggagatttttgtagatgttacaagttccgtggaatgtttggaactgcgagatactgatttggacactttccatttttcagaactatccactggtgaaacaaattcattgattaaaaagtttacatttagaaatgtgaaaatcaccgatgaaagtttgtttcaggttatgaaacttttgaatcagatttctggattgttagaattagagtttgatgactgtacccttaatggagttggtaattttagagcatctgataatgacagagttatagatccaggtaaagtggaaacgttaacaatccggaggctgcatattccaaggttttacttattttatgatctgagcactttatattcacttacagaaagagttaaaagaatcacagtagaaaacagtaaagtttttctggttccttgtttactttcacaacatttaaaatcattagaatacttggatctcagtgaaaatttgatggttgaagaatacttgaaaaattcagcctgtgaggatgcctggccctctctacaaactttaattttaaggcaaaatcatttggcatcattggaaaaaaccggagagactttgctcactctgaaaaacttgactaacattgatatcagtaagaatagttttcattctatgcctgaaacttgtcagtggccagaaaagatgaaatatttgaacttatccagcacacgaatacacagtgtaacaggctgcattcccaagacactggaaattttagatgttagcaacaacaatctcaatttattttctttgaatttgccgcaactcaaagaactttatatttccagaaataagttgatgactctaccagatgcctccctcttacccatgttactagtattgaaaatcagtaggaatgcaataactacgttttctaaggagcaacttgactcatttcacacactgaagactttggaagctggtggcaataacttcatttgctcctgtgaattcctctccttcactcaggagcagcaagcactggccaaagtcttgattgattggccagcaaattacctgtgtgactctccatcccatgtgcgtggccagcaggttcaggatgtccgcctctcggtgtcggaatgtcacaggacagcactggtgtctggcatgtgctgtgctctgttcctgctgatcctgctcacgggggtcctgtgccaccgtttccatggcctgtggtatatgaaaatgatgtgggcctggctccaggccaaaaggaagcccaggaaagctcccagcaggaacatctgctatgatgcatttgtttcttacagtgagcgggatgcctactgggtggagaaccttatggtccaggagctggagaacttcaatccccccttcaagttgtgtcttcataagcgggacttcattcctggcaagtggatcattgacaatatcattgactccattgaaaagagccacaaaactgtctttgtgctttctgaaaactttgtgaagagtgagtggtgcaagtatgaactggacttctcccatttccgtctttttgatgagaacaatgatgctgccattctcattcttctggagcccattgagaaaaaagccattccccagcgcttctgcaagctgcggaagataatgaacaccaagacctacctggagtggcccatggacgaggctcagcgggaaggattttgggtaaatctgagagctgcgataaagtcctag

SEQ ID NO: 24 - protein, 15 aa, Artificial Sequence
  Synthetic
KHLCPSPLFPGPSKP

SEQ ID NO: 25 - DNA, 45 bp, Artificial Sequence
  Synthetic
aaacacctttgtccaagtcccctatttcccggaccttctaagccc

SEQ ID NO: 26 - protein, 38 aa, Artificial Sequence
  Synthetic
SVVDFLPTTAQPTKKSTLKKRVCRLPRPETQKGPLCSP

SEQ ID NO: 27 - DNA, 114 bp, Artificial Sequence
  Synthetic
agtgtggttgatttccttcccaccactgcccagcccaccaagaagtccaccctcaagaagagagtgtgccggttacccaggccagagacccagaagggcccactttgtagcccc

SEQ ID NO: 28 - protein, 22 aa, Artificial Sequence
  Synthetic
EPKSCDKTHTCPPCPAPELLGG

SEQ ID NO: 29 - DNA, 66 bp, Artificial Sequence
  Synthetic
gagcccaaatcttgtgacaaaactcacacatgcccaccgtgcccagcacctgaactcctgggggga

SEQ ID NO: 30 - protein, 76 aa, Artificial Sequence
  Synthetic
AAARWPESPKAQASSVPTAQPQAEGSLAKATTAPATTRNTGRGGEEKKKEKEKEEQEERETKTPECPYVLRWEPSS

SEQ ID NO: 31 - DNA, 228 bp, Artificial Sequence
  Synthetic
gcggccgcacgctggccagagtctccaaaggcacaggcctcctcagtgcccactgcacaaccccaagcagagggcagcctcgccaaggcaaccacagccccagccaccacccgtaacacaggaagaggaggagaagagaagaagaaggagaaggagaaagaggaacaagaagagagagagacaaagacaccagagtgtccgtacgtactgagatgggagccctcgagc

SEQ ID NO: 32 - protein, 201 aa, Artificial Sequence
  Synthetic
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPS

SEQ ID NO: 33 - DNA, 603 bp, Artificial Sequence
  Synthetic
atggtaccacctcccgaaaatgtcagaatgaattctgttaatttcaagaacattctacagtgggagtcacctgcttttgccaaagggaacctgactttcacagctcagtacctaagttataggatattccaagataaatgcatgaatactaccttgacggaatgtgatttctcaagtctttccaagtatggtgaccacaccttgagagtcagggctgaatttgcagatgagcattcagactgggtaaacatcaccttctgtcctgtggatgacaccattattggaccccctggaatgcaagtagaagtacttgctgattctttacatatgcgtttcttagcccctaaaattgagaatgaatacgaaacttggactatgaagaatgtgtataactcatggacttataatgtgcaatactggaaaaacggtactgatgaaaagtttcaaattactccccagtatgactttgaggtcctcagaaacctggagccatggacaacttattgtgttcaagttcgagggtttcttcctgatcggaacaaagctggggaatggagtgagcctgtctgtgagcaaacaacccatgacgaaacggtcccctcc

SEQ ID NO: 34 - protein, 9 aa, Artificial Sequence
  Synthetic
GGGGSGGGS

SEQ ID NO: 35 - DNA, 27 bp, Artificial Sequence
  Synthetic
ggaggtggcggatccggaggtggctcc

SEQ ID NO: 36 - protein, 13 aa, Artificial Sequence
  Synthetic
GGGGSGGGSGGGS

SEQ ID NO: 37 - DNA, 39 bp, Artificial Sequence
  Synthetic
ggaggtggcggatccggaggtggctccggaggtggctcc

SEQ ID NO: 38 - protein, 28 aa, Artificial Sequence
  Synthetic
GGGGSGGGSGGGSSSGGGGSGGGSGGGS

SEQ ID NO: 39 - DNA, 84 bp, Artificial Sequence
  Synthetic
ggaggtggcggatccggaggtggctccggaggtggctcctcgagcggaggtggcggatccggaggtggctccggaggtggctcc

SEQ ID NO: 40 - protein, 8 aa, Artificial Sequence
  Synthetic
SSQPTIPI

SEQ ID NO: 41 - DNA, 24 bp, Artificial Sequence
  Synthetic
tcgagccagcccaccatccccatc

SEQ ID NO: 42 - protein, 57 aa, Artificial Sequence
  Synthetic
VGIIAGLVLFGAVITGAVVAAVMWRRKSSDRKGGSYSQAASSDSAQGSDVSLTACKV

SEQ ID NO: 43 - DNA, 174 bp, Artificial Sequence
  Synthetic
gtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtga

SEQ ID NO: 44 - protein, 27 aa, Artificial Sequence
  Synthetic
FWVLVVVGGVLACYSLLVTVAFIIFWV

SEQ ID NO: 45 - DNA, 81 bp, Artificial Sequence
  Synthetic
ttctgggtgttggtcgttgtgggtggtgtcctggcgtgttattcactgttggttactgtggcttttataattttctgggtg

SEQ ID NO: 46 - protein, 105 aa, Artificial Sequence
  Synthetic
WMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 47 - DNA, 318 bp, Artificial Sequence
  Synthetic
tggatggtggccgtcatcctcatggcctcggtcttcatggtctgcctggcactcctcggctgcttcgccttgctgtggtgcgtttacaagaagacaaagtacgccttctcccctaggaattctcttccacagcacctgaaagagtttttgggccatcctcatcataacacacttctgtttttctcctttccattgtcggatgagaatgatgtttttgacaagctaagtgtcattgcagaagactctgagagcggcaagcagaatcctggtgacagctgcagcctcgggaccccgcctgggcaggggccccaaagctag

SEQ ID NO: 48 - protein, 163 aa, Artificial Sequence
  Synthetic
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - DNA, 492 bp, Artificial Sequence
  Synthetic
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 50 - protein, 898 aa, Artificial Sequence
  Synthetic
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIHGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSLERVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - DNA, 2697 bp, Artificial Sequence
  Synthetic
atgccacatactttgtggatggtgtgggtcttgggggtcatcatcagcctctccaaggaagaatcctccaatcaggcttctctgtcttgtgaccgcaatggtatctgcaagggcagctcaggatctttaaactccattccctcagggctcacagaagctgtaaaaagccttgacctgtccaacaacaggatcacctacattagcaacagtgacctacagaggtgtgtgaacctccaggctctggtgctgacatccaatggaattaacacaatagaggaagattctttttcttccctgggcagtcttgaacatttagacttatcctataattacttatctaatttatcgtcttcctggttcaagcccctttcttctttaacattcttaaacttactgggaaatccttacaaaaccctaggggaaacatctcttttttctcatctcacaaaattgcaaatcctgagagtgggaaatatggacaccttcactaagattcaaagaaaagattttgctggacttaccttccttgaggaacttgagattgatgcttcagatctacagagctatgagccaaaaagtttgaagtcaattcagaatgtaagtcatctgatccttcatatgaagcagcatattttactgctggagatttttgtagatgttacaagttccgtggaatgtttggaactgcgagatactgatttggacactttccatttttcagaactatccactggtgaaacaaattcattgattaaaaagtttacatttagaaatgtgaaaatcaccgatgaaagtttgtttcaggttatgaaacttttgaatcagatttctggattgttagaattagagtttgatgactgtacccttaatggagttggtaattttagagcatctgataatgacagagttatagatccaggtaaagtggaaacgttaacaatccggaggctgcatattccaaggttttacttattttatgatctgagcactttatattcacttacagaaagagttaaaagaatcacagtagaaaacagtaaagtttttctggttccttgtttactttcacaacatttaaaatcattagaatacttggatctcagtgaaaatttgatggttgaagaatacttgaaaaattcagcctgtgaggatgcctggccctctctacaaactttaattttaaggcaaaatcatttggcatcattggaaaaaaccggagagactttgctcactctgaaaaacttgactaacattgatatcagtaagaatagttttcattctatgcctgaaacttgtcagtggccagaaaagatgaaatatttgaacttatccagcacacgaatacacagtgtaacaggctgcattcccaagacactggaaattttagatgttagcaacaacaatctcaatttattttctttgaatttgccgcaactcaaagaactttatatttccagaaataagttgatgactctaccagatgcctccctcttacccatgttgctagtattgaaaatcagtaggaatgcaataactacgttttctaaggagcaacttgactcatttcacacactgaagactttggaagctggtggcaataacttcatttgctcctgtgaattcctctccttcactcaggagcagcaagcactggccaaagtcttgattgattggccagcaaattacctgtgtgactctccatcccatgtgcgtggccagcaggttcaggatgtccgcctctcggtgtcggaatgtcacaggacagcactggtgtctggcatgtgctgtgctctgttcctgctgatcctgctcacgggggtcctgtgccaccgtttccatggcctgtggtatatgaaaatgatgtgggcctggctccaggccaaaaggaagcccaggaaagctcccagcaggaacatctgctatgatgcatttgtttcttacagtgagcgggatgcctactgggtggagaaccttatggtccaggagctggagaacttcaatccccccttcaagttgtgtcttcataagcgggacttcattcatggcaagtggatcattgacaatatcattgactccattgaaaagagccacaaaactgtctttgtgctttctgaaaactttgtgaagagtgagtggtgcaagtatgaactggacttctcccatttccgtctttttgatgagaacaatgatgctgccattctcattcttctggagcccattgagaaaaaagccattccccagcgcttctgcaagctgcggaagataatgaacaccaagacctacctggagtggcccatggacgaggctcagcgggaaggattttgggtaaatctgagagctgcgataaagtccctcgagagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 52 - protein, 803 aa, Artificial Sequence
  Synthetic
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTAAARWPESPKAQASSVPTAQPQAEGSLAKATTAPATTRNTGRGGEEKKKEKEKEEQEERETKTPECPYVLRWEPSSQPTIPILCYLLDGILFIYGVILTALFLRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 2412 bp, Artificial Sequence
  Synthetic
atgccacatactttgtggatggtgtgggtcttgggggtcatcatcagcctctccaaggaagaatcctccaatcaggcttctctgtcttgtgaccgcaatggtatctgcaagggcagctcaggatctttaaactccattccctcagggctcacagaagctgtaaaaagccttgacctgtccaacaacaggatcacctacattagcaacagtgacctacagaggtgtgtgaacctccaggctctggtgctgacatccaatggaattaacacaatagaggaagattctttttcttccctgggcagtcttgaacatttagacttatcctataattacttatctaatttatcgtcttcctggttcaagcccctttcttctttaacattcttaaacttactgggaaatccttacaaaaccctaggggaaacatctcttttttctcatctcacaaaattgcaaatcctgagagtgggaaatatggacaccttcactaagattcaaagaaaagattttgctggacttaccttccttgaggaacttgagattgatgcttcagatctacagagctatgagccaaaaagtttgaagtcaattcagaatgtaagtcatctgatccttcatatgaagcagcatattttactgctggagatttttgtagatgttacaagttccgtggaatgtttggaactgcgagatactgatttggacactttccatttttcagaactatccactggtgaaacaaattcattgattaaaaagtttacatttagaaatgtgaaaatcaccgatgaaagtttgtttcaggttatgaaacttttgaatcagatttctggattgttagaattagagtttgatgactgtacccttaatggagttggtaattttagagcatctgataatgacagagttatagatccaggtaaagtggaaacgttaacaatccggaggctgcatattccaaggttttacttattttatgatctgagcactttatattcacttacagaaagagttaaaagaatcacagtagaaaacagtaaagtttttctggttccttgtttactttcacaacatttaaaatcattagaatacttggatctcagtgaaaatttgatggttgaagaatacttgaaaaattcagcctgtgaggatgcctggccctctctacaaactttaattttaaggcaaaatcatttggcatcattggaaaaaaccggagagactttgctcactctgaaaaacttgactaacattgatatcagtaagaatagttttcattctatgcctgaaacttgtcagtggccagaaaagatgaaatatttgaacttatccagcacacgaatacacagtgtaacaggctgcattcccaagacactggaaattttagatgttagcaacaacaatctcaatttattttctttgaatttgccgcaactcaaagaactttatatttccagaaataagttgatgactctaccagatgcctccctcttacccatgttactagtattgaaaatcagtaggaatgcaataactacgttttctaaggagcaacttgactcatttcacacactgaagactttggaagctggtggcaataacttcatttgctcctgtgaattcctctccttcactcaggagcagcaagcactggccaaagtcttgattgattggccagcaaattacctgtgtgactctccatcccatgtgcgtggccagcaggttcaggatgtccgcctctcggtgtcggaatgtcacaggacagcggccgcacgctggccagagtctccaaaggcacaggcctcctcagtgcccactgcacaaccccaagcagagggcagcctcgccaaggcaaccacagccccagccaccacccgtaacacaggaagaggaggagaagagaagaagaaggagaaggagaaagaggaacaagaagagagagagacaaagacaccagagtgtccgtacgtactgagatgggagccctcgagccagcccaccatccccatcctctgctacctgctggatggaatcctcttcatctatggtgtcattctcactgccttgttcctgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 54 - protein, 434 aa, Artificial Sequence
  Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGSGGGSSSQPTIPIVGIIAGLVLFGAVITGAVVAAVMWRRKSSDRKGGSYSQAASSDSAQGSDVSLTACKV

SEQ ID NO: 55 - DNA, 1305 bp, Artificial Sequence
  Synthetic
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagtacttcgggcagtggtaagcccgggagtggtgagggtagtactaagggtagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggaggtggcggatccggaggtggctccggaggtggctcctcgagccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtga

SEQ ID NO: 56 - protein, 448 aa, Artificial Sequence
  Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGSGGGSSSGGGGSGGGSGGGSSSQPTIPIVGIIAGLVLFGAVITGAVVAAVMWRRKSSDRKGGSYSQAASSDSAQGSDVSLTACK

SEQ ID NO: 57 - DNA, 1350 bp, Artificial Sequence
  Synthetic
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagtacttcgggcagtggtaagcccgggagtggtgagggtagtactaagggtagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggaggtggcggatccggaggtggctccggaggtggctcctcgagcggaggtggcggatccggaggtggctccggaggtggctcctcgagccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtga

SEQ ID NO: 58 - protein, 677 aa, Artificial Sequence
  Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGSGGGSSSMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 59 - DNA, 2034 bp, Artificial Sequence
  Synthetic
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagtacttcgggcagtggtaagcccgggagtggtgagggtagtactaagggtagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggaggtggcggatccggaggtggctccggaggtggctcctcgagcatggtaccacctcccgaaaatgtcagaatgaattctgttaatttcaagaacattctacagtgggagtcacctgcttttgccaaagggaacctgactttcacagctcagtacctaagttataggatattccaagataaatgcatgaatactaccttgacggaatgtgatttctcaagtctttccaagtatggtgaccacaccttgagagtcagggctgaatttgcagatgagcattcagactgggtaaacatcaccttctgtcctgtggatgacaccattattggaccccctggaatgcaagtagaagtacttgctgattctttacatatgcgtttcttagcccctaaaattgagaatgaatacgaaacttggactatgaagaatgtgtataactcatggacttataatgtgcaatactggaaaaacggtactgatgaaaagtttcaaattactccccagtatgactttgaggtcctcagaaacctggagccatggacaacttattgtgttcaagttcgagggtttcttcctgatcggaacaaagctggggaatggagtgagcctgtctgtgagcaaacaacccatgacgaaacggtcccctcctggatggtggccgtcatcctcatggcctcggtcttcatggtctgcctggcactcctcggctgcttcgccttgctgtggtgcgtttacaagaagacaaagtacgccttctcccctaggaattctcttccacagcacctgaaagagtttttgggccatcctcatcataacacacttctgtttttctcctttccattgtcggatgagaatgatgtttttgacaagctaagtgtcattgcagaagactctgagagcggcaagcagaatcctggtgacagctgcagcctcgggaccccgcctgggcaggggccccaaagctag

SEQ ID NO: 60 - protein, 435 aa, Artificial Sequence
  Synthetic
METDTLLLWVLLLWVPSTGDIVLTQSPASLAVSLGQRATISCRASQSVSTSSYSYMHWYQQKPGQPPKLLIKYASNLESGVPARFSGSGSGTDFTLNIHPVEEEDTATYYCQHSWEIPYTFGGGTKLEIKRGSTSGSGKPGSGEGSTKGQIQLVQSGPELKKPGETVKISCKASGYTFTDYSMHWVKQAPGKGLKWMGWINTETGEPTYADDFKGRFAFSLETSASTAYLQINNLKNEDTATYFCARGKYGAFAYWGQGTLVTVSAGSEQKLISEEDLGSTTTTPAPRPPTPAPTIASQPLSLRPEACRPAAGGAVHTRGLDFACDISSFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQ

SEQ ID NO: 61 - DNA, 1336 bp, Artificial Sequence
  Synthetic
cgtctagagcaatggagacagacacactcctgctatgggtgctgctgctctgggttccatccactggtgacattgtgctaacacagtctcctgcttccttagctgtatctctggggcagagggccaccatctcatgcagggccagccaaagtgtcagtacatctagctatagttatatgcactggtatcaacagaaaccaggacagccacccaaactcctcatcaagtatgcttccaacctagaatctggggtccctgccaggttcagtggcagtgggtctgggacagacttcaccctcaacatccatcctgtggaggaggaggatactgcaacatattactgtcagcacagttgggagattccgtacacgttcggaggggggaccaagctggaaataaaacggggatcaacttcgggcagtggtaagcctggtagtggtgagggtagtaccaagggccagatccagttggtgcagtctggacctgagctgaagaagcctggagagacagtcaaaatctcctgcaaggcttctggttataccttcacagactattcaatgcactgggtgaagcaggctccaggaaagggtttaaagtggatgggctggataaacactgagactggtgagccaacgtatgcagatgacttcaagggacggtttgccttctctttggaaacctctgccagcactgcctatttgcagatcaacaacctcaaaaatgaggacacggctacatatttctgtgctagaggaaagtatggggcctttgcttactggggccaagggactctggtcactgtctcagcaggatccgaacagaaactgatctctgaggaggacctggggtcgactaccacaacgccagctccccgcccaccaacgcctgcgccaactattgcctcacagcctttgagtctccggccagaagcatgtcgccccgctgccggtggagcagtccatacaagaggccttgacttcgcgtgcgatatctcgagcttctgggtgttggtcgttgtgggtggtgtcctggcgtgttattcactgttggttactgtggcttttataattttctgggtgaggagtaagaggagcaggctcctgcacagtgactacatgaacatgactccccgccgcccagggccaacccgcaagcattaccagccctatgccccaccacgcgacttcgcagcctataggtctcaagttagaaaagcagctataacatcttatgagaaatctgatggagtatatacagggctcagcacgcgaaatcaggagacctatgaaactctgaagcatgagaagcccccgcagtagggcggccgcgaattcgc

SEQ ID NO: 62 - protein, 439 aa, Artificial Sequence
  Synthetic
MRFSAQLLGLLVLWIPSTADIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPWTFGGGTKLEIKGSTSGSGKPGSGEGSTKGEVKLVESGGGLVQPGGSLRLSCATSGFTFTDYYMSWVRQPPGKALEWLGFIRNKANGYTTEYSASVKGRFTISRDNSQNILYLQMNTLRAEDSATYYCARVTGTHWYFDVWGAGTTVSHRLGSEQKLISEEDLGSTTTTPAPRPPTPAPTIASQPLSLRPEACRPAAGGAVHTRGLDFACDISSFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQ

SEQ ID NO: 63 - DNA, 1348 bp, Artificial Sequence
  Synthetic
cgtctagaacaatgaggttctctgctcagcttctggggctgcttgtgctctggataccttccacagcagatattgtgatgacgcaggctgcattctccaatccagtcactcttggaacatcagcttccatctcatgcaggtctagtaagagtctcctacatagtaatggcatcacttatttgtattggtatctacagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctcgaacttccgtggacgttcggtggaggcaccaagctggaaatcaaaggatcaacttcgggcagtggtaagcctggtagtggtgagggtagtaccaagggcgaggtgaagctggtggagtctggaggaggcttggtacagcctgggggttctctgagactctcctgtgcaacttctgggttcaccttcactgattactacatgagctgggtccgccagcctccaggaaaggcacttgagtggttgggttttattagaaacaaagctaatggttacacaacagagtacagtgcatctgtgaagggtcggttcaccatctccagagataattcccaaaacatcctctatcttcaaatgaacaccctgagagctgaggacagtgccacttattactgtgcaagagttactgggacgcactggtacttcgatgtctggggcgcagggaccacggtgtctcaccgtctcggatccgaacagaaactgatctctgaggaggacctggggtcgactaccacaacgccagctccccgcccaccaacgcctgcgccaactattgcctcacagcctttgagtctccggccagaagcatgtcgccccgctgccggtggagcagtccatacaagaggccttgacttcgcgtgcgatatctcgagcttctgggtgttggtcgttgtgggtggtgtcctggcgtgttattcactgttggttactgtggcttttataattttctgggtgaggagtaagaggagcaggctcctgcacagtgactacatgaacatgactccccgccgcccagggccaacccgcaagcattaccagccctatgccccaccacgcgacttcgcagcctataggtctcaagttagaaaagcagctataacatcttatgagaaatctgatggagtatatacagggctcagcacgcgaaatcaggagacctatgaaactctgaagcatgagaagcccccgcagtagggcggccgcgaattcgc

SEQ ID NO: 64 - protein, 898 aa, Artificial Sequence; identical to SEQ ID NO: 50
  Synthetic

SEQ ID NO: 65 - DNA, 2697 bp, Artificial Sequence; identical to SEQ ID NO: 51
  Synthetic

SEQ ID NO: 66 - protein, 803 aa, Artificial Sequence; identical to SEQ ID NO: 52
  Synthetic

SEQ ID NO: 67 - DNA, 2412 bp, Artificial Sequence; identical to SEQ ID NO: 53
  Synthetic

SEQ ID NO: 68 - protein, 15 aa, Artificial Sequence
  Synthetic
GSEQKLISEEDLGST

SEQ ID NO: 69 - DNA, 45 bp, Artificial Sequence
  Synthetic
ggatccgaacagaaactgatctctgaggaggacctggggtcgact

(19) United States
(12) Patent Application Publication
(10) Pub. No.: US 2022/0186232 A1
(43) Pub. Date: Jun. 16, 2022

(54) GENETICALLY REPROGRAMMED TREGS EXPRESSING CARS

(71) Applicant: Gavish-Galilee Bio Applications Ltd., Kiryat Shmona (IL)

(72) Inventors: Gideon Gross, Moshav Almagor (IL); Hadas Weinstein-Marom, Upper Galilee (IL); Sarah Pozner, Tzfat (IL); Amit Kroner, Kibbutz Nir David (IL)

(73) Assignee: Gavish-Galilee Bio Applications Ltd., Kiryat Shmona (IL)

(21) Appl. No.: 17/598,208

(22) PCT Filed: Mar. 26, 2020
(86) PCT No.: PCT/IL2020/050360

§ 371 Date: Sep. 24, 2021

Related U.S. Application Data
(60) Provisional application No. 62/823,711, filed on Mar. 26, 2019.
(60) Provisional application No. 62/898,471, filed on Sep. 10, 2019.

(51) Int. Cl. C12N 15/62; A61P 1/00; C07K 14/54; C07K 14/725; C07K 16/12

(52) U.S. Cl. CPC C12N 15/62; A61P 1/00; A61K 2039/5156; C07K 14/7051; C07K 16/12; C07K 14/5428

(57) ABSTRACT

Nucleic acid molecules comprising a nucleotide sequence encoding an activating chimeric antigen receptor (aCARs) are provided, said aCARs comprising (i) an extracellular binding-domain specifically binding an antigen selected from an antigen of the commensal gut microflora and a self-cell surface antigen specific to the lamina propria (LP) or submucosa of the gastrointestinal tract; (ii) a transmembrane domain; (iii) an intracellular domain including at least one signal transduction element that activates and/or co-stimulates a T cell; and optionally (iv) a stalk region linking the extracellular domain and the transmembrane domain. Compositions and vectors comprising the nucleic acid molecules encoding the aCAR as well as methods for preparing regulatory T cells comprising the vectors and expressing the aCARs are further provided as are methods for treating or preventing a disease, disorder or condition manifested in excessive activity of the immune system in a subject, comprising administering to said subject the mammalian Treg expressing on its surface an aCAR. The regulatory T cells optionally express a membrane-bound homodimeric IL-10 conferring a stable Tr1 phenotype.

### FIELD OF THE INVENTION

The present invention relates in general to genetically reprogrammed regulatory T cells optionally expressing membrane-bound IL-10 and their use in inducing either systemic or tissue-restricted immunosuppression and treating diseases manifested in excessive activity of the immune system.

### BACKGROUND

Harnessing CD4 regulatory T cells (Tregs) for suppressing local inflammation and restoring immunological balance holds great promise in the treatment of pathologies as diverse as autoimmune diseases, inflammatory bowel diseases, allergies, atherosclerosis, transplant rejection, graft-versus-host disease and more. However, Tregs, either natural (nTregs) or induced (iTregs), including type 1 regulatory T cells (Tr1 cells) form only a minor fraction in the entire human CD4 T cell population. Consequently, there is an urgent need for the development of Treg-based therapies designed for recruiting, inducing, or engineering autologous or allogeneic Tregs at adequate numbers and stable phenotype which are critical for clinical efficacy and safety of treatment.

An important subtype of iTregs, the type 1 or Tr1 cells are induced in the periphery in a TCR- and antigen-specific manner upon chronic exposure to antigen on dendritic cells in the presence of interleukin 10 (IL-10). Tr1 cells are characterized by a non-proliferative (anergic) state, high production of IL-10 and TGF-β and the ability to suppress effector T cells (Teffs) in a cell-to-cell contact-independent manner. A recent study demonstrated that the enforced constitutive expression of IL-10 in human CD4 T cells, accomplished by lentiviral transduction, was sufficient for endowing these cells with a particularly stable Tr1 phenotype in an autocrine fashion (1). Although providing an elegant solution to de-novo generation of Tr1 cells, this protocol results in Tr1 cells that produce IL-10 constitutively, in an activation-independent manner. In the clinical setting this uncontrolled IL-10 secretion poses the risk of systemic and prolonged immune suppression, losing the intended antigen- or tissue-selectivity of the therapeutic effects exerted by the Tr1 cells.

There remains therefore a pressing need for efficient Treg—and in particular—efficient Tr1 immunotherapies for autoimmune disease and other autoimmune-related disorders.

### SUMMARY OF INVENTION

In one aspect, the present invention provides a nucleic acid molecule comprising a nucleotide sequence encoding an activating chimeric antigen receptor (aCAR) comprising (i) an extracellular binding-domain specifically binding an antigen selected from an antigen of the commensal gut microflora and a self-cell surface antigen specific to the lamina propria (LP) or submucosa of the gastrointestinal tract; (ii) a transmembrane domain; (iii) an intracellular domain including at least one signal transduction element that activates and/or co-stimulates a T cell; and optionally (iv) a stalk region linking the extracellular domain and the transmembrane domain.

In certain embodiments, in addition to the nucleotide sequence encoding an aCAR, the nucleic acid molecule further comprises a nucleotide sequence encoding a homodimeric IL-10 that is linked to a transmembrane-intracellular stretch, optionally through a flexible hinge.

In an additional aspect, the present invention provides a composition comprising the nucleic acid molecule comprising a nucleotide sequence encoding an aCAR of the present invention but is lacking the nucleotide sequence encoding a homodimeric IL-10.

In another aspect, the composition comprises the nucleic acid molecule comprising a nucleotide sequence encoding an aCAR of the present invention and a nucleotide sequence encoding a homodimeric IL-10 as defined herein that is linked to a transmembrane-intracellular stretch, optionally through a flexible hinge.

In a further aspect, the present invention provides a composition comprising a first nucleic acid molecule comprising a nucleotide sequence encoding an aCAR of the present invention and a second physically separate nucleic acid molecule comprising a nucleotide sequence encoding a homodimeric IL-10 as defined herein that is linked to a transmembrane-intracellular stretch, optionally through a flexible hinge.

In yet an additional aspect, the present invention provides a vector, such as a viral vector, comprising any one of the nucleic acid molecules as defined herein.

In yet another aspect, the present invention provides a composition comprising at least one vector, such as a viral vector, wherein the composition comprises one vector of the present invention; or said composition comprises at least two vectors, wherein one of the vectors comprises the nucleic acid molecule comprising a nucleotide sequence encoding an aCAR of the present invention and another vector comprises the nucleic acid molecule comprising a nucleotide sequence encoding a homodimeric IL-10 as defined herein.

In yet a further aspect, the present invention provides a mammalian regulatory T cell (Treg) comprising any of the nucleic acid molecules of the present invention, or the vector, optionally integrated into the genome of the cell, as defined herein.

In still an additional aspect, the present invention provides a method of preparing allogeneic or autologous Tregs, the method comprising contacting CD4 T cells with the nucleic acid molecule comprising a nucleotide sequence encoding an aCAR of the present invention alone or in combination with a nucleotide sequence encoding a homodimeric IL-10 as defined herein, a retroviral vector comprising it, or a composition according to any one of the above embodiments, thereby preparing allogeneic or autologous Tregs expressing on their surface aCARs with or without mem-IL-10.

In still another aspect, the present invention provides a method of treating or preventing a disease, disorder or condition in a subject, comprising administering to said subject the mammalian Treg expressing on its surface an aCAR alone or in combination with a homodimeric IL-10 as defined herein, wherein said disease, disorder or condition is manifested in excessive activity of the immune system, such as an autoimmune disease, allergy, asthma, and organ and bone marrow transplantation.

### BRIEF DESCRIPTION OF DRAWINGS

FIG. 1 depicts a schematic presentation of membrane-anchored homodimeric IL-10.

FIGS. 2A-D show analysis of membrane-anchored homodimeric IL-10 (memIL-10) expression in T cells and its effect on IL-10 receptor (IL-10R) and CD49b. Human Jurkat or primary, peripheral blood lymphocyte-derived CD4 T cells (A, B) and mouse B3Z or NOD splenic CD4 T cells (C, D) were electroporated with 10 μg of in-vitro transcribed mRNA encoding human or mouse memIL-10, respectively. Cells were analyzed by flow cytometry 24 hours (A-C) or 48 hours (D, left and right) post-transfection. Human or mouse memIL-10 and IL-10R and human CD49b were analyzed by monoclonal antibodies specific to the respective human or mouse proteins, respectively.

FIGS. 3A-D depict schematic presentations of native IL-10 homodimer bound to its cell surface receptor (A) and of the three membrane-anchored derivatives of IL-10 (mem-IL-10): (B) mem-IL-10 with short linker; (C) mem-IL-10 with long linker; and (D) mem-IL-10 linked to IL-10Rβ (IL-10Rβ fusion).

FIG. 4 shows cell surface expression of the three memIL-10 derivatives in Jurkat cells 24 hours post-mRNA electroporation. Human Jurkat CD4 T cells were electroporated with 10 μg of each of the indicated mRNAs (sL and lL stand for short and long linker, respectively). Twenty four hours cells were analyzed by flow cytometry for surface expression of IL-10.

FIGS. 5A-C show that memIL-10 expression in CD4 T cells induces spontaneous phosphorylation of STAT3. Mouse CD4 T cells were either electroporated with irrelevant mRNA (Irr. mRNA), mRNA encoding short linker memIL-10 (sLmemIL-10), long linker memIL-10 (lLmemIL-10) or IL-10 linked to the IL-10Rβ chain (memIL-10Rβ) or treated with soluble recombinant IL-10 (sIL-10) at 20 ng/ml. Twenty four hours later cells were subjected to flow cytometry analysis for surface IL-10 (A), surface IL-10Rα chain (B) or intracellularly for phosphorylated STAT3 (pSTAT3) (C).

FIGS. 6A-B show analysis of retrovirally transduced mouse CD4 T cells expressing memIL-10. Phenotypic analysis of short-linker memIL-10-transduced mouse CD4 T cells (v-memIL-10), 48 hours (A) and 6 days (B) post-transduction. Analysis was performed in parallel on memIL-10(+) and memIL-10(−) cells growing in the same cell culture, staining for LAG-3, CD49b and PD-1. As a positive control non-transduced cells were treated with soluble IL-10 (sIL-10). Mock, cells treated with identical protocol as retrovirally transduced cells but without exposure to viral particles.

FIG. 7 shows secretion of IL-10 by activated, memIL-10 transduced mouse CD4 T cells. Cells from the same experiment as in FIG. 6 were stimulated by an anti-TCR-CD3 mAb (2C11) and their growth medium was subjected to an IL-10 ELISA. Mock- and Green Fluorescent Protein (GFP)-transduced T cells served as negative controls.

FIGS. 8A-C show phenotypic characterization of memIL-10 transduced human CD4 T cells. CD4 T cells were isolated by magnetic beads from peripheral blood mononuclear cells prepared from a blood sample of a healthy donor. Cells were grown in the presence of the anti-CD3 and anti-CD28 antibodies and IL-2 to the desired number and transduced with recombinant retrovirus encoding memIL-10 or an irrelevant gene (Irr.), or treated with soluble IL-10 (sIL-10). Cells were grown in the presence of IL-2 and samples were taken for flow cytometry analysis for the indicated cell surface markers at day 1 (A), day 5 (B) and day 18 (C). At day 18 non-transduced Tregs were added to the analysis for comparison of cell surface markers. At each time point cells expressing memIL-10 (Pos, solid frame)) were analyzed side by side with cells from the same culture, which do not express IL-10 (Neg, dotted frame).

FIG. 9 shows a second experiment phenotyping memIL-10-transduced human CD4 T cells. Cells were prepared and transduced with memIL-10 and analyzed 4 days later for the indicated markers as described in the legend to FIG. 8. Non-transduced (Naïve) and mock-transduced (Mock) CD4 cells served as negative controls. MemIL-10 positive cells were compared to memIL-10 negative cells from the same culture as well as to naïve CD4 T cells grown in the presence of 50, 100 or 300 ng/ml sIL-10. Shown are % of positively stained cell in each sample. Double pos, % of cells stained positive for LAG-3 and CD49b.

FIGS. 10A-C depict schematic representations of three types of anti-peptidoglycan (PGN) Chimeric Antigen Receptors (CARs) (A) and their surface expression (B, C). The CAR constructs shown in (A, left) and (A, middle) are based on TLR2 while (A, right) presents a conventional CAR. Heavy chain variable domain, V_{H}; light chain variable domain, V_{L}; single chain variable fragment, ScFv; Toll/interleukin-1 receptor domain, TIR; *, inactivating mutation in the TIR domain of TLR2. (B) Flow cytometry analysis for TLR2 expression of MCF7 cells transfected with mRNA encoding the TLR-2-based CARs. Human THP-1 cells, which naturally express TLR-2, served as a positive control (P.C.). (C) Flow cytometry analysis for Myc tag expression by K652 cells transfected with mRNA encoding anti-PGN conventional CARs.

FIG. 11 depicts the linear arrangement of the different members of the aCAR. Tag (in this case Myc tag), T.

FIG. 12 shows the results of an ELISA testing binding of two anti-PGN monoclonal antibodies (mAb), 3C11 (mouse IgG, purified from hybridoma) and 3F6 (mouse IgM, hybridoma supernatant), to PGN. OD 450, Optical Density at 450 nm; Irr. Ab, control irrelevant IgG.

FIG. 13 shows PGN-specific activation of anti-PGN CAR-T cells. B3Z T cells carrying the nuclear factor of activated T cells (NFAT)-LacZ reporter gene for T cell activation were transfected with mRNA encoding each of the two anti-PGN CARs (CAR-3C11 and CAR-3F6) or GFP as a control. Cells were then incubated overnight in the presence or absence of PGN from S. aureus. Results are presented as OD of the colorimetric chlorophenol red-β-D-galactopyranoside (CPRG) assay for β-Gal activity. Anti-PGN CAR prepared from the 3C11 hybridoma, 1564; anti-PGN CAR from 3F6, 1565.

FIG. 14 shows B3Z reporter T cells electroporated with mRNA encoding the two anti-PGN CARs (CAR-3C11 and CAR-3F6) and controls and cultured in the presence of PGN derived from Gram-negative or Gram-positive bacteria. 24 hours later cells were subjected to the colorimetric CPRG reporter assay for T cell activation. Anti-PGN CAR prepared from the 3C11 hybridoma, 1564; anti-PGN CAR from 3F6, 1565; non-productive CAR from 3F6, 1566; An irrelevant CAR, negative control; S. aureus PGN, SA; E. coli PGN, EK.

### DETAILED DESCRIPTION

One specific treatment in which CD4 regulatory T cells (Tregs) hold great therapeutic promise is the treatment of inflammatory bowel diseases (IBD), namely, Crohn's disease (CD) and ulcerative colitis (UC). IBD are thought to result from an inappropriate inflammatory response to microbial components following injury of the intestinal epithelial barrier in genetically susceptible individuals (2). Harnessing Tregs to selectively suppress chronic inflammation and restore intestinal homeostasis is widely explored as treatment for IBD (3-5). Yet, progress in this field suffers from general lack of information on genuine T cell antigens associated with pathogenesis and the general elusiveness of Treg specificity.

While the use of dietary antigens as Treg targets has been considered (6), the inventors of the present invention found that constituents of the commensal gut microflora, such as lipopolysaccharide (LPS), peptidoglycan and lipopeptide, which can traverse the epithelial layer to the lamina propria (LP) and gut-associated lymphoid tissue (GALT), are more relevant clinically. Although there is evidence that these substances can exit the LP, their systemic concentrations are very low (7-9). In particular, peptidoglycan (PGN) is a major polymeric cell wall component of both Gram-positive and Gram-negative bacteria, which is sensed by different cells comprising the gut barrier, either intracellularly by NOD2 (10, 11) or extracellularly by TLR2 (12, 13).

There is now compelling evidence that engagement of Tregs with antigen through their endogenous TCR is critical for immune suppression in-vivo (14). Yet, the selection of genuine T cell antigens that are associated with the above-mentioned disorders which can be presented to CD4 Tregs as peptide/HLA-II complexes is limited. Moreover, conventional strategies for targeting such complexes by adequate numbers of Tregs are HLA-II-dependent and can be tremendously laborious (see, for example, the expansion of autologous OVA-specific Tregs for the treatment of Crohn's Disease (6)). In contrast, the approach of the present invention is based on the well-established ability to genetically redirect T cells against cell surface antigens of choice using chimeric antigen receptors, or CARs. CARs were originally developed by one of the inventors at the late 1980's (15) and nowadays are mostly used in cancer immunotherapy for the selective targeting of tumors by Teff cells (16).

It has been found in accordance with the present invention that two different anti-PGN CARs activate T cells in a PGN-dependent manner and that PGN from Gram-negative and Gram-positive bacteria were equally effective in activating the T cells.

Thus, in one aspect, the present invention provides a nucleic acid molecule comprising a nucleotide sequence encoding an activating chimeric antigen receptor (aCAR) comprising (i) an extracellular binding-domain specifically binding an antigen selected from an antigen of the commensal gut microflora and a self-cell surface antigen specific to the lamina propria (LP) or submucosa of the gastrointestinal tract; (ii) a transmembrane domain; (iii) an intracellular domain including at least one signal transduction element that activates and/or co-stimulates a T cell; and optionally (iv) a stalk region linking the extracellular domain and the transmembrane domain.

In a certain embodiment, in addition to the nucleotide sequence encoding an aCAR, the nucleic acid molecule further comprises a nucleotide sequence encoding a homodimeric IL-10 that is linked to a transmembrane-intracellular stretch, optionally through a flexible hinge, also referred to herein as mem-IL-10. The mem-IL-10 and methods for producing and using it are disclosed in WO 2019/180724, incorporated by reference as if fully disclosed herein.

In certain embodiments, the nucleic acid molecule comprises a nucleotide sequence encoding the aCAR of the present invention but is lacking the nucleotide sequence encoding a homodimeric IL-10.

Any relevant technology may be used to engineer a recognition moiety/binding domain that confers to the aCAR specific binding to its targets. In certain embodiments, the extracellular domain comprises (i) an antibody, derivative or fragment thereof, such as a humanized antibody; a human antibody; a functional fragment of an antibody; a single-domain antibody, such as a Nanobody; a recombinant antibody; and a single chain variable fragment (ScFv); (ii) an extracellular domain of a TLR, derivative or fragment thereof (in the case of TLR-ligands); (iii) an antibody mimetic, such as an affibody molecule; an affilin; an affimer; an affitin; an alphabody; an anticalin; an avimer; a DARPin; a fynomer; a Kunitz domain peptide; and a monobody; or (iv) an aptamer.

In principle, methods for preparing new scFvs against TLR ligands of choice are readily available to the person of skill in the art and can e.g. be selected using Ab display technologies (17).

In certain embodiments, the antigen of the commensal gut microflora that the extracellular binding domain of the aCAR specifically binds is an antigen of the mammalian, in particular the human, gastrointestinal microbiota, also known as gut flora or gut microbiota, which are the microorganisms that live a non-harmful coexistence in the digestive tracts of mammals, such as humans.

In certain embodiments, the antigen of the commensal gut microflora is an antigen of anaerobic bacteria, which represent over 99% of the gut bacteria.

In certain embodiments, the antigen of the commensal gut microflora is an antigen of a bacterium belonging to one of the four dominant bacterial phyla in the human gut: Firmicutes, Bacteroidetes, Actinobacteria, and Proteobacteria, and in particular of a bacterium of the genus Bacteroides, Clostridium, Faecalibacterium, Eubacterium, Ruminococcus, Peptococcus, Peptostreptococcus, Bifidobacterium, Escherichia or Lactobacillus.

In certain embodiments, the antigen is a toll-like receptor (TLR)-ligand antigen of the commensal gut microflora, such as a ligand of TLR1, TLR2, TLR4, TLRS, TLR6, TLR9 and TLR10.

In certain embodiments the extracellular domain of the TLR is, or is derived from, the extracellular domain of a mammal TLR, such as the extracellular domain of a human TLR.

In certain embodiments, the TLR-ligand antigen that the binding domain binds is selected from Table 1.

TABLE 1
TLR LIGANDS

| Receptor | Ligand(s) | Ligand location |
|---|---|---|
| TLR 1 | multiple triacyl lipopeptides | Bacterial |
| | | lipoprotein |
| TLR 2 | multiple glycolipids | Bacterial |
| | | peptidoglycans |
| | multiple lipopeptides and proteolipids | Bacterial |
| | | peptidoglycans |
| | diacyl lipopeptides, such as lipoteichoic acid | Gram-positive |
| | | bacteria |
| | HSP70 | Host cells |
| | viral products, among them hepatitis C core and NS3 protein from | Host cells |
| | the hepatitis C virus and glycoprotein B from cytomegalovirus | |
| | zymosan (Beta-glucan) | Fungi |
| TLR 4 | lipopolysaccharide | Gram-negative |
| | | bacteria |
| | several heat shock proteins | Bacteria and host |
| | | cells |
| | fibrinogen | host cells |
| | heparan sulfate fragments | host cells |
| | hyaluronic acid fragments | host cells |
| TLR 5 | Bacterial flagellin | Bacteria |
| | Profilin | Toxoplasma |
| | | gondii |
| | loxoribine (a guanosine analogue) | |
| | bropirimine | |
| | resiquimod | |
| | single-stranded RNA | RNA viruses |
| TLR6 | diacyl lipopeptides, such as lipoteichoic acid | Bacteria |
| | macrophage-activating lipopeptide | Mycoplasma |
| | fungal ligands such as glucuronoxylomannan, phospholipomannan | Fungus |
| | and zymosan | |
| | protozoan ligand - lipopeptidophosphoglycan | protozoa |
| TLR 9 | unmethylated CpG Oligodeoxynucleotide DNA | Bacteria, DNA |
| | | viruses |
| TLR 10 | triacylated lipopeptides | |
| TLR 11 | Profilin | Toxoplasma |
| | | gondii |
| TLR 12 | Profilin | Toxoplasma |
| | | gondii |
| TLR 13 | bacterial ribosomal RNA sequence ″CGGAAAGACC″ (but not | Virus, bacteria |
| | the methylated version) [SEQ ID NO: 1] | |

In certain embodiments, the antigen is selected from peptidoglycan; a lipopeptide, such as a triacyl lipopeptide; lipoteichoic acid; lipopolysaccharide (LPS); flagellin; bacterial CpG-containing DNA and viral CpG-containing DNA.

Peptidoglycan, also known as murein, is a polymer consisting of sugars and amino acids that forms a mesh-like layer outside the plasma membrane of bacteria (but not Archaea), forming the cell wall. The sugar component consists of alternating residues of β-(1,4) linked N-acetylglucosamine and N-acetylmuramic acid. Attached to the N-acetylmuramic acid is a peptide chain of three to five amino acids. It is a ligand of TLR2 and thus in certain embodiments, the extracellular binding domain is a TLR 2 binding domain, derivative or fragment thereof, preferably a human TLR 2 binding domain, derivative or fragment thereof. Alternatively, the extracellular binding domain is an antibody, derivative or fragment thereof (e.g. an scFv) capable of specific binding of peptidoglycan. Examples of such antibodies is Peptidoglycan Monoclonal Antibody, Clone 3F6B3, LifeSpan BioSciences, 3C11 (ATCC® HB-8511™), IgG1(κ) and 3F6 (ATCC® HB-8512™), IgM(κ), from which an anti-peptidoglycan scFv is readily cloned.

Non-limiting examples of lipopeptides that the extracellular binding domain binds are PAM2Cys, PAM3Cys, O-Palmitoyl-Ser, N′-Palmitoyl-Lys, Lipoamino acids (LAAs) and Dipalmitylglutamic acid) (Taguchi. Micro and Nanotechnology in Vaccine Development. Micro and Nano Technologies 2017, Pages 149-170. Chapter Eight—Nanoparticle-Based Peptide Vaccines https://www.sciencedirect.com/topics/medicine-and-dentistry/lipopeptide. See Table 2).

TABLE 2
EXAMPLES OF LIPOPEPTIDES

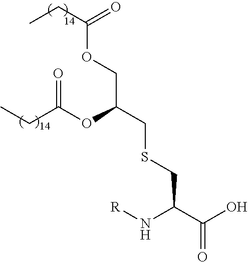

Pam₂Cys: R = H
Pam₃Cys: R = CH₃(CH₂)₁₄CO

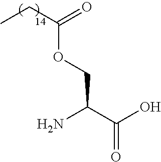

O-Palmitoyl-Ser

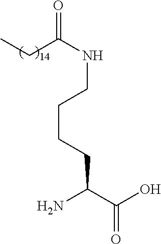

N′-Palmitoyl-Lys

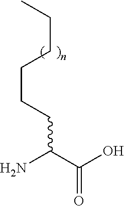

Lipoaminoacids (LAAs)
n = 1-11

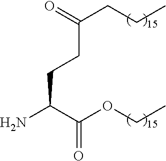

Dipalmitylglutamic acid

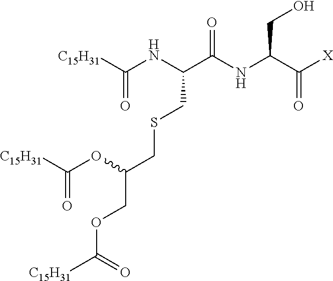

Generic Pam₃Cys-based lipopeptide structure
where X indicates a peptide sequence

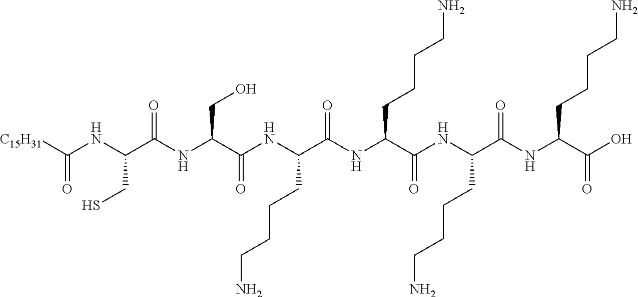

PamCSK4

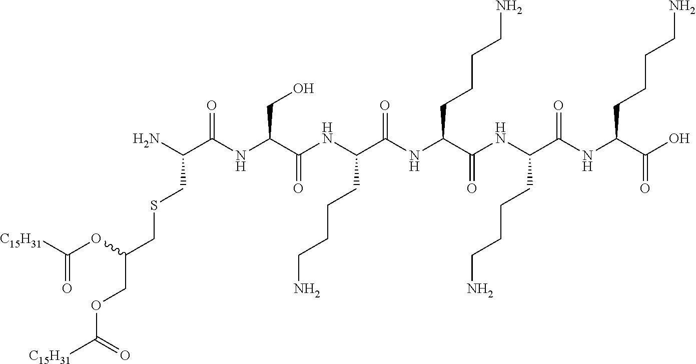

Pam₂CSK4

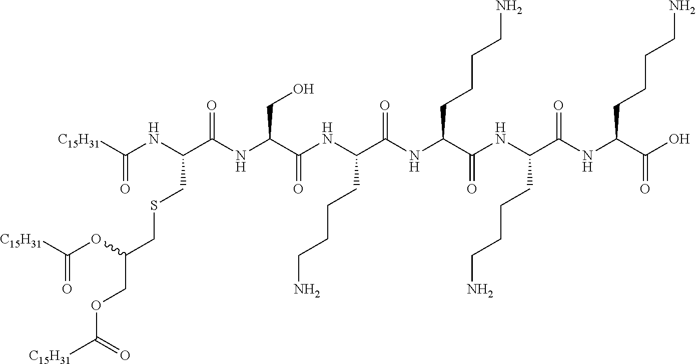

Pam₃CSK4

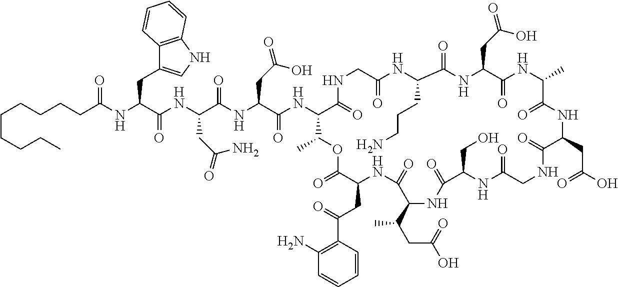 [SMILES: CCCCCCCCCC(=O)N[C@@H](Cc1c[nH]c2ccccc12)C(=O)N[C@@H](CC(N)=O)C(=O)N[C@@H](CC(=O)O)C(=O)N[C@@H]1C(=O)NCC(=O)N[C@@H](CCCN)C(=O)N[C@@H](CC(=O)O)C(=O)N[C@H](C)C(=O)N[C@@H](CC(=O)O)C(=O)NCC(=O)N[C@H](CO)C(=O)N[C@@H]([C@@H](C)CC(=O)O)C(=O)N[C@@H](CC(=O)c2ccccc2N)C(=O)O[C@@H]1C]

Daptomycin (an example of cyclic lipopeptide)

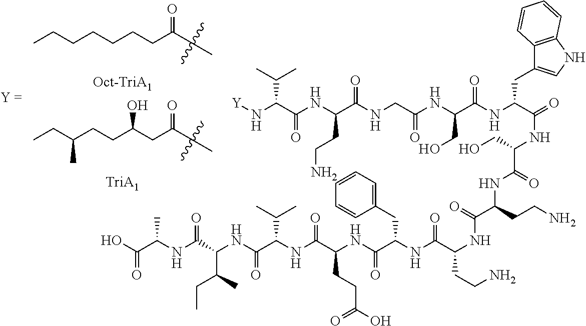

Tridecaptin analogs TriA₁ and Oct-TriA₁

Lipoteichoic acid (LTA) is a major constituent of the cell wall of gram-positive bacteria. The structure of LTA varies between the different species of Gram-positive bacteria and may contain long chains of ribitol or glycerol phosphate. It is a ligand of TLR2 and thus in certain embodiments, the extracellular binding domain is a TLR 2 binding domain, derivative or fragment thereof, preferably a human TLR 2 binding domain, derivative or fragment thereof. Alternatively, the extracellular binding domain is an antibody, derivative or fragment thereof (e.g. an scFv) capable of specific binding of LTA. One such antibody is anti-lipoteichoic acid (LTA) mAb, clone 55, LifeSpan BioSciences, from which an anti-LTA scFv is readily cloned.

Lipopolysaccharides, also known as lipoglycans and endotoxins, are large molecules consisting of a lipid and a polysaccharide composed of O-antigen, outer core and inner core joined by a covalent bond; they are found in the outer membrane of Gram-negative bacteria. The O-antigen is a repetitive glycan polymer contained within the LPS. The O antigen is attached to the core oligosaccharide, and comprises the outermost domain of the LPS molecule. The Core domain always contains an oligosaccharide component that attaches directly to lipid A and commonly contains sugars such as heptose and 3-Deoxy-D-manno-oct-2-ulosonic acid (also known as KDO, keto-deoxyoctulosonate). The LPS Cores of many bacteria also contain non-carbohydrate components, such as phosphate, amino acids, and ethanolamine substituents. The term lipopolysaccharide as used herein refers also to lipooligosaccharide (“LOS”), a low-molecular-weight form of lipopolysaccharide. It is a ligand of TLR4 and thus in certain embodiments, the extracellular binding domain is a TLR 4 binding domain, derivative or fragment thereof, preferably a human TLR 4 binding domain, derivative or fragment thereof. Alternatively, the extracellular binding domain is an antibody, derivative or fragment thereof (e.g. an scFv) capable of specific binding of LPS. One such antibody is anti-LPS mAb, clone NYRChlam LPS, LifeSpan BioSciences, from which an anti-LPS scFv is readily cloned.

Flagellin is the subunit protein which polymerizes to form the filaments of bacterial flagella and is present in large amounts on nearly all flagellated bacteria. It is a ligand of TLRS and thus in certain embodiments, the extracellular binding domain is a TLR 5 binding domain, derivative or fragment thereof, preferably a human TLR 5 binding domain, derivative or fragment thereof. Alternatively, the extracellular binding domain is an antibody, derivative or fragment thereof (e.g. an scFv) capable of specific binding of flagellin. One such antibody is anti-flagellin mAb, clone FLIC-1, LifeSpan BioSciences, from which an anti-flagellin scFv is readily cloned.

The term “CpG-containing DNA” as used herein refers to CpG oligodeoxynucleotides, short single-stranded synthetic DNA molecules that contain a cytosine triphosphate deoxynucleotide (“C”) followed by a guanine triphosphate deoxynucleotide (“G”). It is a ligand of TLR9 and 10 and thus in certain embodiments, the extracellular binding domain is a TLR 9 or 10 binding domain, derivative or fragment thereof, preferably a human TLR 9 or 10 binding domain, derivative or fragment thereof. Alternatively, the extracellular binding domain is an antibody, derivative or fragment thereof (e.g. an scFv) capable of specific binding of CpG-containing DNA.

In certain embodiments, the extracellular binding-domain of the aCAR is selected from an extracellular domain of TLR1, TLR2, TLR4, TLR5, TLR6, TLR9 or TLR10, or derivative or fragment thereof; and a single chain variable fragment (scFv) specifically binding said antigen.

In certain embodiments, the extracellular binding domain binds peptidoglycans from a variety of Gram-negative and Gram-positive bacteria.

In certain embodiments, the extracellular binding domain is a scFv specifically binding peptidoglycan, such as but not limited to an scFv derived from a monoclonal antibody binding PGNs from a variety of Gram-negative and Gram-positive bacteria, such as 3C11 (ATCC® HB-8511™), IgG1 (κ) and 3F6 (ATCC® HB-8512™), IgM(κ).

In certain embodiments, the scFv is derived from the monoclonal antibody 3C11 and comprises a light chain variable domain (V_{L}) set forth in SEQ ID NO: 3 (also including the leader peptide and encoded by e.g. a nucleic acid molecule as set forth in SEQ ID NO: 4), connected to a heavy chain variable domain (V_{H}) of SEQ ID NO: 7 (encoded by e.g. a nucleic acid molecule as set forth in SEQ ID NO: 8), optionally through a first flexible linker, e.g. of the amino acid sequence GSTSGSGKPGSGEGSTKG (SEQ ID NO: 5), encoded by e.g. a nucleic acid molecule as set forth in SEQ ID NO: 6.

In certain embodiments, the scFv is derived from the monoclonal antibody 3F6 and comprises a light chain variable domain (V_{L}) of SEQ ID NO: 16 (also including the leader peptide and encoded by e.g. a nucleic acid molecule as set forth in SEQ ID NO: 17), connected to a heavy chain variable domain (V_{H}) of SEQ ID NO: 18 (encoded by e.g. a nucleic acid molecule as set forth in SEQ ID NO: 19), optionally through a first flexible linker, e.g. of the amino acid sequence GSTSGSGKPGSGEGSTKG (SEQ ID NO: 5), encoded by e.g. a nucleic acid molecule as set forth in SEQ ID NO: 6.

In certain embodiments, the extracellular binding domain binding peptidoglycan is a TLR 2 binding domain, preferably a human TLR 2 binding domain of the sequence set forth in SEQ ID NO; 20 (e.g. encoded by the DNA sequence of SEQ ID NO: 21).

The role of the intracellular domain of the aCAR is to provide T cell activating signals upon binding of the binding domain to its specific antigen. In accordance with the present invention, these antigens are T cell antigens associated with pathogenesis and the aCAR is designed to redirect Tregs to tissue exhibiting these antigens, to activate the Tregs and subdue excessive Teff activity. The intracellular domain is thus designed to activate Tregs, such as Tr1 T cells, and any signal transduction element (activating or costimulatory) or combination of signal transduction elements that activate T cells in general and Tregs in particular can be used, whether known today or yet to be discovered. Similarly, any linker, flexible hinge or stalk and transmembrane domain or sequence can be used according to the present invention as long as it contributes to an efficiently expressed and functioning aCAR. A comprehensive review of the different building blocks commonly used in aCARs that are readily applicable in the aCARs of the present invention is found e.g. in Dotti et al. (18) and Guedan etal. (19).

In certain embodiments, the intracellular domain of the aCAR, regardless of the nature of its binding domain, comprises at least one domain which is homologous to an immunoreceptor tyrosine-based activation motif (ITAM) of for example, CD3ζ (zeta), CD3 η (eta) chain, or FcRγ chains; to a Toll/interleukin-1 receptor (TIR) domain of for example TLR1, TLR2, TLR4, TLR5, TLR6, TLR9 or TLR10; or to a co-stimulatory signal transduction element of for example, B cell receptor polypeptide, CD27, CD28, CD278 (ICOS), CD137 (4-1BB), CD134 (OX40), Dap10, CD2, CD5, ICAM-1, LFA-1, Lck, TNFR-I, TNFRII, Fas, CD30, or combinations thereof. Additional intracellular domains will be apparent to those of skill in the art and may be used in connection with alternate embodiments of the invention.

In a certain embodiment, the intracellular domain of the aCAR, regardless of the nature of its binding domain, comprises a tandem arrangement of signal transduction elements selected from TIR, a co-stimulatory signal transduction element of CD28 and an ITAM of FcRγ (also referred to herein as signal transduction elements of TIR-CD28-FcRγ), wherein the TIR is derived from TLR1, TLR2, TLR4, TLR5, TLR6, TLR9 or TLR10; and a tandem arrangement of a co-stimulatory signal transduction element of CD28 and an ITAM of FcRγ (also referred to herein as signal transduction elements of CD28-FcRγ).

The transmembrane domain of the CAR, regardless of the nature of its binding and intracellular domains, may comprise the transmembrane sequence from any protein which has a transmembrane domain, including any of the type I, type II or type III transmembrane proteins, or an artificial hydrophobic sequence. The transmembrane domains of the CARs of the invention may be selected so as not to dimerize. Additional transmembrane domains will be apparent to those of skill in the art and may be used in connection with alternate embodiments of the invention.

In certain embodiments, the transmembrane domain of the aCAR is selected from the transmembrane domain of CD28 (e.g. human CD28 as set forth in SEQ ID NO: 44; e.g. encoded by a nucleotide sequence as set forth in SEQ ID NO: 45), CD3-zeta, TLR1, TLR2, TLR4, TLR5, TLR6, TLR9, TLR10 and Fc receptor.

In certain embodiments, the aCAR comprises a stalk region linking the extracellular domain and the transmembrane domain, which may include Fc fragments of antibodies or fragments or derivatives thereof, hinge regions of antibodies or fragments or derivatives thereof, CH2 regions of antibodies, CH3 regions of antibodies, artificial spacer sequences or combinations thereof. For example, the stalk may include peptide spacers such as Gly₃ or CH1, CH2 and CH3 domains of IgGs, such as human IgG4.

In certain embodiments, regardless of the nature of its binding and transmembrane domains, the stalk region is selected from the stalk or hinge of CD28 (SEQ ID NO: 24; e.g. encoded by a nucleotide sequence as set forth in SEQ ID NO: 25), CD8α (for example as set forth in SEQ ID NO: 9; e.g. encoded by a nucleotide sequence as set forth in SEQ ID NO: 10), CD8β (for example as set forth in SEQ ID NO: 26; e.g. encoded by a nucleotide sequence as set forth in SEQ ID NO: 27) and the heavy chain of IgG (for example as set forth in SEQ ID NO: 28; e.g. encoded by a nucleotide sequence as set forth in SEQ ID NO: 29) or IgD (for example as set forth in SEQ ID NO: 30; e.g. encoded by a nucleotide sequence as set forth in SEQ ID NO: 31.

In particular embodiments, the antigen is a TLR-ligand antigen of the commensal gut microflora; said intracellular domain comprises at least one domain which is homologous to ITAM of for example, CD3ζ, CD3η chain, or FcRγ chains; to a TIR of for example TLR1, TLR2, TLR4, TLR5, TLR6, TLR9 or TLR10; or to a co-stimulatory signal transduction element of for example, B cell receptor polypeptide, CD27, CD28, CD278 (ICOS), CD137 (4-1BB), CD134 (OX40), Dap10, CD2, CD5, ICAM-1, LFA-1, Lck, TNFR-I, TNFRII, Fas, CD30, or combinations thereof; said transmembrane domain is selected from a transmembrane region of a Type I transmembrane protein, an artificial hydrophobic sequence, the transmembrane domain of CD28, CD3ζ, TLR1, TLR2, TLR4, TLR5, TLR6, TLR9 or TLR10, and Fc receptor; and the aCAR comprises a stalk region linking the extracellular domain and the transmembrane domain, and said stalk region is selected from the stalk or hinge of CD28, CD8α, CD8β and the heavy chain of IgG or IgD.

In particular embodiments, the TLR-ligand antigen is selected from a ligand of TLR1, TLR2, TLR4, TLR5, TLR6, TLR9 and TLR10; and said intracellular domain comprises a tandem arrangement of signal transduction elements selected from signal transduction elements of TIR-CD28-FcRγ, wherein the TIR is derived from TLR1, TLR2, TLR4, TLR5, TLR6, TLR9 or TLR10; and signal transduction elements of CD28-FcRγ.

In particular embodiments, the TLR-ligand antigen is selected from peptidoglycan; a lipopeptide, such as a triacyl lipopeptide; lipoteichoic acid; lipopolysaccharide; flagellin; bacterial CpG-containing DNA and viral CpG-containing DNA.

In particular embodiments, the extracellular binding-domain is selected from an extracellular domain of TLR1, TLR2, TLR4, TLR5, TLR6, TLR9 or TLR10, or derivative or fragment thereof; and an scFv specifically binding said TLR-ligand antigen.

In particular embodiments, the extracellular binding-domain is an scFv that specifically binds peptidoglycan or an extracellular domain of TLR2.

In a certain embodiment, the aCAR comprises an scFv specifically binding PGN, a stalk region comprising the hinge of CD8α, a transmembrane domain comprising the transmembrane domain of CD28, and an intracellular domain comprising a tandem arrangement of signal transduction elements of CD28-FcRγ.

In certain embodiments, the aCAR comprises a complete TLR, such as a complete TLR2, and the intracellular domain comprises CD3ζ and the intracellular domain of TLR2 with wild-type TIR or the TIR incapacitated by an inactivating mutation (Pro681His mutation in human TLR2 (20) or corresponding to it in other species' TLR2). Alternatively, the aCAR comprises the extracellular binding domain of a TLR, such as TLR2, and the signal transduction element of CD3ζ, e.g. in the form of the complete intracellular domain of CD3ζ.

In a certain embodiment, the aCAR comprises a TLR, such as TLR2, and the intracellular domain comprises a tandem arrangement of signal transduction elements of CD28-FcRγ linked to the TIR domain of said TLR, optionally comprising the inactivating mutation.

In a certain embodiment, the homodimeric IL-10 comprises a first and a second IL-10 monomer connected in a single-chain configuration such that the C-terminus of the first IL-10 monomer is linked to the N-terminus of the second IL-10 monomer via a first flexible linker.

Flexible peptide linkers are well-known in the art. Empirical linkers designed by researchers are generally classified into three categories according to their structures: flexible linkers, rigid linkers, and in vivo cleavable linkers as defined e.g. in (21-23), each one of which is incorporated by reference as if fully disclosed herein.

As stated above, the first linker is a flexible linker and its structure is selected from any one of the linkers disclosed in (21-23). In principle, to provide flexibility, the linkers are generally composed of small, non-polar (e.g. Gly) or polar (e.g. Ser or Thr) amino acids, such an underlying sequence of alternating Gly and Ser residues. Solubility of the linker and associated homodimeric IL-10 may be enhanced by including charged residues; e.g. two positively charged residues (Lys) and one negatively charged residue (Glu). The linker may vary from 2 to 31 amino acids, optimized for each condition so that the linker does not impose any constraints on the conformation or interactions of the linked partners in lengths, such as between 12 and 18 residues.

In a certain embodiment, the first flexible linker has the amino acid sequence GSTSGSGKPGSGEGSTKG [SEQ ID NO: 5], as encoded by a nucleotide sequence e.g. as set forth in SEQ ID NO: 6.

In certain embodiments, the homodimeric IL-10 is linked to the transmembrane-intracellular stretch via a flexible hinge, and the flexible hinge comprises a polypeptide selected from a hinge region of CD8α (for example as set forth in SEQ ID NO: 9; e.g. encoded by a nucleotide sequence as set forth in SEQ ID NO: 10), a hinge region of CD28 for example as set forth in SEQ ID NO: 24; e.g. encoded by a nucleotide sequence as set forth in SEQ ID NO: 25), a hinge region of CD8β for example as set forth in SEQ ID NO: 26; e.g. encoded by a nucleotide sequence as set forth in SEQ ID NO: 27), a hinge region of a heavy chain of IgG (for example as set forth in SEQ ID NO: 28; e.g. encoded by a nucleotide sequence as set forth in SEQ ID NO: 29), a hinge region of a heavy chain of IgD (for example as set forth in SEQ ID NO: 30; e.g. encoded by a nucleotide sequence as set forth in SEQ ID NO: 31); an extracellular stretch of an IL-10R β chain (as set forth in SEQ ID NO: 32; e.g. encoded by a nucleotide sequence as set forth in SEQ ID NO: 33); and a second flexible linker comprising an amino acid spacer of up to 28 amino acids, e.g. comprising one Gly₄Ser(Gly₃Ser) sequence (SEQ ID NO: 34; for example encoded by a nucleotide sequence as set forth in SEQ ID NO: 35), or two Gly₄Ser(Gly₃Ser) sequences with one or two Ser residues inserted between them.

In certain embodiments, the second flexible linker comprises a 21 amino acid sequence comprising the amino acid sequence Gly₄Ser(Gly₃Ser)₂ (referred to herein as “short linker”; SEQ ID NO: 36; for example encoded by a nucleotide sequence as set forth in SEQ ID NO: 37).

In certain embodiments, the second flexible linker consists of a 28 amino acid spacer comprising the amino acid sequence Gly₄Ser(Gly₃Ser)₂Ser₂(Gly₃Ser)₃ (referred to herein as “long linker”; SEQ ID NO: 38; for example encoded by a nucleotide sequence as set forth in SEQ ID NO: 39) and the connecting peptide of SEQ ID NO: 40, for example encoded by a nucleotide sequence as set forth in SEQ ID NO: 41.

In certain embodiments, the second flexible linker of any one of the above embodiments further comprises an 8 amino acid bridge of the sequence SSQPTIPI (referred to herein as “connecting peptide”; SEQ ID NO: 40; for example encoded by a nucleotide sequence as set forth in SEQ ID NO: 41) derived from the membrane-proximal part of the connecting peptide of HLA-A2.

In certain embodiments, the transmembrane-intracellular stretch of the mem-IL-10 is derived from the heavy chain of a human MHC class I molecule selected from an HLA-A, HLA-B or HLA-C molecule, preferably HLA-A2 (as set forth in SEQ ID NO: 42; e.g. encoded by a nucleotide sequence as set forth in SEQ ID NO: 43); human CD28 (as set forth in SEQ ID NO: 44; e.g. encoded by a nucleotide sequence as set forth in SEQ ID NO: 45); or human IL-10R β chain (as set forth in SEQ ID NO: 46; e.g. encoded by a nucleotide sequence as set forth in SEQ ID NO: 47).

In certain embodiments, the amino acid sequence of the complete mem-IL-10 comprises or essentially consists of the homodimeric IL-10 linked via the short second flexible linker and the connecting peptide to the transmembrane-intracellular stretch of HLA-A2 as set forth in SEQ ID NO: 54; e.g. encoded by a nucleotide sequence as set forth in SEQ ID NO: 55.

In certain embodiments, the amino acid sequence of the complete mem-IL-10 comprises or essentially consists of the homodimeric IL-10 linked via the long second flexible linker and the connecting peptide to the transmembrane-intracellular stretch of HLA-A2 as set forth in SEQ ID NO: 56; e.g. encoded by a nucleotide sequence as set forth in SEQ ID NO: 57).

In certain embodiments, the mem-IL-10 is fused to the IL-10Rβ extracellular domain (for example as set forth in SEQ ID NO: 32) via a second flexible linker, and optionally further to the IL-10Rβ transmembrane & cytosolic domains (for example as set forth in SEQ ID NO: 46), e.g. encoded by a nucleotide sequence as set forth in SEQ ID NO: 47.

In certain embodiments, the mem-IL-10 is fused to the N-terminus of an essentially complete IL-10R β chain via the short linker (as set forth in SEQ ID NO: 46; e.g. encoded by a nucleotide sequence as set forth in SEQ ID NO: 47).

In particular embodiments, the homodimeric IL-10 comprises a first and a second IL-10 monomer connected in a single-chain configuration such that the C-terminus of the first IL-10 monomer is linked to the N-terminus of the second IL-10 monomer via a first flexible linker; said homodimeric IL-10 is linked to the transmembrane-intracellular stretch via a flexible hinge, and said flexible hinge comprises a polypeptide selected from a hinge region of CD8α, a hinge region of a heavy chain of IgG, a hinge region of a heavy chain of IgD; an extracellular stretch of an IL-10R β chain; and a second flexible linker comprising an amino acid spacer of up to 28 amino acids, such as a 21 amino acid spacer consisting of one Gly4Ser(Gly3Ser)2 sequence [SEQ ID NO: 36] and an additional 8 amino acid bridge of the sequence SSQPTIPI [SEQ ID NO: 40]; and said transmembrane-intracellular stretch of said homodimeric IL-10 is derived from the heavy chain of a human MHC class I molecule selected from an HLA-A, HLA-B or HLA-C molecule, preferably HLA-A2; or the IL-10R β chain.

TABLE 3
SEQUENCE IDENTIFICATION NUMBERS (SEQ ID NOS. OR SIN)

| SIN | SEQUENCE NAME/DOMAIN | SEQUENCE TYPE |
|---|---|---|
| 1 | TRL13 ligand | RNA |
| 2 | 5′′ untranslated sequence of 3C11/3F6 | DNA |
| 3 | Leader peptide-VL (3C11) | PROT |
| 4 | Leader peptide-VL (3C11) | DNA |
| 5 | First flexible linker | PROT |
| 6 | First flexible linker | DNA |
| 7 | VH(3C11) | PROT |
| 8 | VH(3C11) | DNA |
| 9 | hinge region of CD8α | PROT |
| 10 | hinge region of CD8α | DNA |
| 11 | CD28 transmembrane & intracellular | PROT |
| 12 | CD28 transmembrane & intracellular | DNA |
| 13 | FcRγ intracellular | PROT |
| 14 | FcRγ intracellular | DNA |
| 15 | 3′ untranslated sequence of 3C11/3F6 | DNA |
| 16 | Leader peptide-V_{L} (3F6) | PROT |
| 17 | Leader peptide-V_{L} (3F6) | DNA |
| 18 | V_{H}(3F6) | PROT |
| 19 | V_{H}(3F6) | DNA |
| 20 | human TLR2 extracellular binding domain | PROT |
| 21 | human TLR2 extracellular binding domain | DNA |
| 22 | full human TLR2 | PROT |
| 23 | full human TLR2 | DNA |
| 24 | CD28 hinge (stalk) | PROT |
| 25 | CD28 hinge (stalk) | DNA |
| 26 | hinge region of CD8β | PROT |
| 27 | hinge region of CD8β | DNA |
| 28 | hinge region of the heavy chain of IgG1 | PROT |
| 29 | hinge region of the heavy chain of IgG1 | DNA |
| 30 | Human IgD hinge protein | PROT |
| 31 | Human IgD hinge protein | DNA |
| 32 | extracellular stretch of the IL-10R β chain | PROT |
| 33 | extracellular stretch of the IL-10R β chain | DNA |
| 34 | second flexible linker min sequence | PROT |
| 35 | second flexible linker min sequence | DNA |
| 36 | short linker | PROT |
| 37 | short linker | DNA |
| 38 | long linker | PROT |
| 39 | long linker | DNA |
| 40 | connecting peptide | PROT |
| 41 | connecting peptide | DNA |
| 42 | HLA-A2 transmembrane-intracellular stretch peptide | PROT |
| 43 | HLA-A2 transmembrane-intracellular stretch peptide | DNA |
| 44 | CD28 transmembrane peptide | PROT |
| 45 | CD28 transmembrane peptide | DNA |
| 46 | IL-10Rβ transmembrane & cytosolic domain | PROT |
| 47 | IL-10Rβ transmembrane & cytosolic domain | DNA |
| 48 | Human CD3 zeta | PROT |
| 49 | Human CD3 zeta | DNA |
| 50 | TLR2-TIR (*) zeta (Pro681His mutation) | PROT |
| 51 | TLR2-TIR (*) zeta (Pro681His mutation) | DNA |
| 52 | TLR2 IgD zeta protein | PROT |
| 53 | TLR2 IgD zeta protein | DNA |
| 54 | Complete sequence mem-IL10 HLA/short linker | PROT |
| 55 | Complete sequence mem-IL10 HLA/short linker | DNA |
| 56 | Complete sequence mem-IL10HLA/long linker | PROT |
| 57 | Complete sequence mem-IL10HLA/long linker | DNA |
| 58 | Complete sequence mem-IL10/IL10-Rβ/short linker | PROT |
| 59 | Complete sequence mem-IL10/IL10-Rβ/short linker | DNA |
| 60 | Complete sequence aCAR | PROT |
| | (3C11)/CD8a/CD28transmem + intracellular/FcRg | |
| 61 | Complete sequence aCAR 5′ | DNA |
| | UT/(3C11)/CD8a/CD28transmem + intracellular/FcRg/3′ UT | |
| 62 | Complete sequence aCAR | PROT |
| | (3F6)/CD8a/CD28transmem + intracellular/FcRg | |
| 63 | Complete sequence aCAR 5' | DNA |
| | UT/(3F6)/CD8a/CD28transmem + intracellular/FcRg/3′ UT | |
| 64 | Complete sequence aCAR TLR2-TIR(*)-zeta CARs | PROT |
| 65 | Complete sequence aCAR TLR2-TIR(*)-zeta CARs | DNA |
| 66 | Complete sequence aCAR TLR2-IgD-zeta | PROT |
| 67 | Complete sequence aCAR TLR2-IgD-zeta | DNA |
| 68 | Myc tag | PROT |
| 69 | Myc tag | DNA |

In particular embodiments, the first flexible linker has the amino acid sequence GSTSGSGKPGSGEGSTKG [SEQ ID NO: 5].

In particular embodiments, the homodimeric IL-10 is linked to the N-terminus of the essentially complete IL-10R β chain.

Non-limiting examples of aCARs and mem-IL-10 constructs are disclosed in the Examples section. The sequence ID numbers (SIN) of the amino acid sequences of the domains of these constructs and the nucleic acid sequences encoding them are disclosed in Table 3.

The polypeptides making up the aCAR or mem-IL-10 of the present invention that are encoded by the nucleic acid molecules of the invention are not limited to those defined herein by specific amino acid sequences but may also be variants or homologs of these oligopeptides or have amino acid sequences that are substantially identical to those disclosed above. A “substantially identical” amino acid sequence as used herein refers to a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid with another of the same class, e.g., substitution of one hydrophobic amino acid with another hydrophobic amino acid, a polar amino acid with another polar amino acid, a basic amino acid with another basic amino acid and an acidic amino acid with another acidic amino acid. One or more amino acids can be deleted from the peptide, thus obtaining a fragment thereof without significantly altering its biological activity.

In certain embodiments, the amino acid sequence of the complete membrane-bound IL-10 or each one of the various sub-regions of the membrane-bound IL-10 as disclosed above i.e. the homodimeric IL-10 in which the first and second IL-10 monomers are connected in a single-chain configuration via a first flexible linker; the first flexible linker per se, the flexible hinge; and the transmembrane-intracellular stretch, is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% identical to a relevant sequence set forth in one of the SEQ ID NOs. in Table 3, such as SEQ ID NOs: 5, 9, 28, 30, 32, 34, 36, 38, 40, 42, 44, 46, 54, 56 and 58.

In certain embodiments, the amino acid sequence of the complete membrane-bound IL-10 or each one of the various sub-regions of the membrane-bound IL-10 as disclosed above i.e. the homodimeric IL-10 in which the first and second IL-10 monomers are connected in a single-chain configuration via a first flexible linker; the first flexible linker per se, the flexible hinge; and the transmembrane-intracellular stretch, as well as the whole construct, is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98, or 99% identical to a relevant sequence set forth in one of the SEQ ID NOs. in Table 3, such as SEQ ID NOs: 5, 9, 28, 30, 32, 34, 36, 38, 40, 42, 44, 46, 54, 56 and 58.

In certain embodiments, the isolated nucleic acid molecule comprises a polynucleotide sequence encoding the complete membrane-bound IL-10 or each one of the various sub-regions of the membrane-bound IL-10 as disclosed above i.e. the homodimeric IL-10 in which the first and second IL-10 monomers are connected in a single-chain configuration via a first flexible linker; the first flexible linker per se, the flexible hinge; and the transmembrane-intracellular stretch, as well as the whole construct, that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% identical to a relevant sequence set forth in one of the SEQ ID NOs. in Table 3, such as SEQ ID NOs: 6, 10, 29, 31, 33, 35, 37, 39, 41, 43, 45, 47, 55, 57 and 59.

In certain embodiments, the isolated nucleic acid molecule comprises a polynucleotide sequence encoding the complete membrane-bound IL-10 or each one of the various sub-regions of the membrane-bound IL-10 as disclosed above i.e. the homodimeric IL-10 in which the first and second IL-10 monomers are connected in a single-chain configuration via a first flexible linker; the first flexible linker per se, the flexible hinge; and the transmembrane-intracellular stretch, as well as the whole construct is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98, or 99% identical to a relevant sequence set forth in one of the SEQ ID NOs. in Table 3, such as 6, 10, 29, 31, 33, 35, 37, 39, 41, 43, 45, 47, 55, 57 and 59.

In certain embodiments, the isolated nucleic acid molecule comprises a polynucleotide sequence encoding the complete membrane-bound IL-10 or each one of the various sub-regions of the membrane-bound IL-10 as disclosed above i.e. the homodimeric IL-10 in which the first and second IL-10 monomers are connected in a single-chain configuration via a first flexible linker; the flexible linker per se, the flexible hinge; and the transmembrane-intracellular stretch, as well as the whole construct as set forth in one of SEQ ID NOs: 6, 10, 29, 31, 33, 35, 37, 39, 41, 43, 45, 47, 55, 57 and 59.

In certain embodiments, the amino acid sequence of the complete CAR or each one of its various sub-regions or combinations thereof, i.e. the V_{L} and V_{H} domains of anti-PGN scFv (derived from 3C11 or 3F6), in which the V_{L} and V_{H} domains are connected in a single-chain configuration via a first flexible linker; the flexible linker per se, human TLR2 binding domain or the complete human TLR2 molecule, CD8α hinge, IgD hinge, CD28 transmembrane domain, intracellular domain comprising at least one signal transduction element of e.g. TIR, CD28, FcRγ or CD3ζ, wherein the TIR is derived from TLR2 or is inactivated, is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% identical to a relevant sequence set forth in one of the SEQ ID NOs. in Table 3, such as SEQ ID NOs: 3, 5, 7, 9, 11, 13, 16, 18, 20, 22, 24, 26, 28, 30, 34, 36, 38, 40, 42, 44, 46, 48, 50, 52, 60, 62, 64 and 66.

In certain embodiments, the amino acid sequence of the complete CAR or each one of its various sub-regions or combinations thereof, i.e. the VL and VH domains of anti-PGN scFv (derived from 3C11 or 3F6), in which the VL and VH domains are connected in a single-chain configuration via a first flexible linker; the flexible linker per se, human TLR2 binding domain or the complete human TLR2 molecule, CD8α hinge, IgD hinge, CD28 transmembrane domain, and intracellular domain comprising at least one signal transduction elements of e.g. TIR, -CD28, -FcRγ or CD3ζ, wherein the TIR is derived from TLR2 or is inactivated, or signal transduction elements of CD28-FcRγ, is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98, or 99% identical to a relevant sequence set forth in one of the SEQ ID NOs. in Table 3, such as SEQ ID NOs: 3, 5, 7, 9, 11, 13, 16, 18, 20, 22, 24, 26, 28, 30, 34, 36, 38, 40, 42, 44, 46, 48, 50, 52, 60, 62, 64 and 66.

In certain embodiments, the isolated nucleic acid molecule comprises a polynucleotide sequence encoding the complete CAR or each one of its various sub-regions the VL and VH domains of anti-PGN scFv (derived from 3C11 or 3F6), in which the VL and VH domains are connected in a single-chain configuration via a first flexible linker; the flexible linker per se, human TLR2 binding domain or the complete human TLR2 molecule, CD8α hinge, IgD hinge, CD28 transmembrane domain, and intracellular domain comprising at least one signal transduction elements of e.g. TIR, -CD28, -FcRγ or CD3ζ, wherein the TIR is derived from TLR2 or is inactivated, or signal transduction elements of CD28-FcRγ, is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% identical to a relevant sequence set forth in one of the SEQ ID NOs. in Table 3, such as SEQ ID NOs: 2, 4, 6, 8, 10, 12, 14, 15, 17, 19, 21, 23, 25, 27, 29, 31, 35, 37, 39, 41, 43, 45, 47, 49, 51, 53, 61, 63, 65 and 67.

In certain embodiments, the isolated nucleic acid molecule comprises a polynucleotide sequence encoding the complete CAR or each one of its various sub-regions the VL and VH domains of anti-PGN scFv (derived from 3C11 or 3F6), in which the VL and VH domains are connected in a single-chain configuration via a first flexible linker; the flexible linker per se, human TLR2 binding domain or the complete human TLR2 molecule, CD8α hinge, IgD hinge, CD28 transmembrane domain, and intracellular domain comprising at least one signal transduction elements of e.g. TIR, -CD28, -FcRγ or CD3ζ, wherein the TIR is derived from TLR2 or is inactivated, or signal transduction elements of CD28-FcRγ, is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98, or 99% identical to a relevant sequence set forth in one of the SEQ ID NOs. in Table 3, such as SEQ ID NOs: 2, 4, 6, 8, 10, 12, 14, 15, 17, 19, 21, 23, 25, 27, 29, 31, 35, 37, 39, 41, 43, 45, 47, 49, 51, 53, 61, 63, 65 and 67.

In certain embodiments, the isolated nucleic acid molecule comprises a polynucleotide sequence encoding the complete CAR or each one of its various sub-regions the VL and VH domains of anti-PGN scFv (derived from 3C11 or 3F6), in which the VL and VH domains are connected in a single-chain configuration via a first flexible linker; the flexible linker per se, human TLR2 binding domain or the complete human TLR2 molecule, CD8α hinge, IgD hinge, CD28 transmembrane domain, and intracellular domain comprising at least one signal transduction elements of e.g. TIR, -CD28, -FcRγ or CD3ζ, wherein the TIR is derived from TLR2 or is inactivated, or signal transduction elements of CD28-FcRγ, as set forth in one of SEQ ID NOs: 2, 4, 6, 8, 10, 12, 14, 15, 17, 19, 21, 23, 25, 27, 29, 31, 35, 37, 39, 41, 43, 45, 47, 49, 51, 53, 61, 63, 65 and 67.

In an additional aspect, the present invention provides a composition comprising the nucleic acid molecule comprising a nucleotide sequence encoding an aCAR according to any one of the above embodiments but is lacking the nucleotide sequence encoding a homodimeric IL-10.

In another aspect, the composition comprises the nucleic acid molecule comprising a nucleotide sequence encoding an aCAR according to any one of the above embodiments and a nucleotide sequence encoding a homodimeric IL-10 that is linked to a transmembrane-intracellular stretch, optionally through a flexible hinge according to any one of the above embodiments.

In a further aspect, the present invention provides a composition comprising a first nucleic acid molecule comprising a nucleotide sequence encoding an aCAR according to any one of the above embodiments and a second physically separate nucleic acid molecule comprising a nucleotide sequence encoding a homodimeric IL-10 that is linked to a transmembrane-intracellular stretch, optionally through a flexible hinge according to any one of the above embodiments.

The nucleic acid molecules of the present invention are delivered into T cells using any well-known method in the field: For example, Matuskova and Durinikova (24) teach that there are two systems for the delivery of transgenes into a cell—viral and non-viral. The non-viral approaches are represented by polymer nanoparticles, lipids, calcium phosphate, electroporation/nucleofection or biolistic delivery of DNA-coated microparticles.

There are two main types of vectors that can be used in accordance with the present invention depending on whether the DNA is integrated into chromatin of the host cell or not. Retroviral vectors such as those derived from gammaretroviruses or lentiviruses persist in the nucleus as integrated provirus and reproduce with cell division. Other types of vectors (e.g. those derived from herpesviruses or adenoviruses) remain in the cell in the episomal form.

Thus, in yet an additional aspect, the present invention provides a vector, such as a viral vector, comprising any one of the nucleic acid molecules described above.

Examples of vectors include but are not limited to viral vectors, such as lentiviral vectors (e.g. self-inactivating (SIN) lentiviral vectors), retroviral vectors, foamy virus vectors, adenovirus, adeno-associated virus (AAV) vectors, pox virus, alphavirus, and herpes virus, hybrid vectors or plasmid transposons (for example sleeping beauty transposon system) or integrase-based vector systems. Other vectors that may be used in connection with alternate embodiments of the invention will be apparent to those of skill in the art.

Viruses of the Retroviridae or Retrovirus family, which includes the gamma-retrovirus and lentivirus genera, such as the murine stem cell virus, Moloney murine leukemia virus, bovine leukaemia virus, Rous sarcoma virus, and spumavirus, have the unique ability to integrate permanently into the host genome and thereby enable long-term stable gene expression. In fact, of the 52 clinical trials evaluating CAR-T cell in solid tumors which are listed in (25), 24 use retroviral vectors and 9 use lentiviral vectors. It is also noted that the two FDA-approved CAR products for the treatment of B cell malignancies are Kymriah™ (lentiviral vector) and Yescarta™ (gamma-retroviral vector). Thus, good candidates for the viral vector of the present invention may be retroviral vectors, lentiviral vectors and gamma-retroviral vectors. For example, the retrovirus may be derived from Moloney murine leukemia virus or murine stem cell virus sequences (gamma-retroviral vectors).

Retroviral vectors are often provided as ‘split-vector systems’ in which viral genes and transgenes are separated across several plasmids. The most commonly used viral vector systems are made up of separate envelope and packaging plasmids as well as transfer plasmids. This concept ensures safe handling and expression of these vectors. Thus, the term “viral vector” as used herein refers to a single vector as well as to two or more vectors.

In certain embodiments, the nucleic acid molecule comprises a single polypeptide-encoding nucleotide sequence encoding the aCAR of the present invention, or two polypeptide-encoding nucleotide sequences, one encoding the aCAR of the present invention and the second encoding the mem-IL-10 as defined above, i.e. the nucleic acid molecule of the viral vector does not encode for additional different proteins, but may comprise additional control elements such as promoters and terminators.

In certain embodiments, the nucleotide sequence per se or of the vector's nucleic acid molecule comprises an internal ribosome entry site (IRES) between the nucleotide sequence encoding for the aCAR and the nucleotide sequence encoding for the homodimeric IL-10.

In certain embodiments, the nucleotide sequence per se or of the vector's nucleic acid molecule comprises a viral self-cleaving 2A peptide between the nucleotide sequence encoding for the aCAR and the nucleotide sequence encoding for the homodimeric IL-10. In particular the viral self-cleaving 2A peptide may be selected from the group consisting of T2A from Thosea asigna virus (TaV), F2A from Foot-and-mouth disease virus (FMDV), E2A from Equine rhinitis A virus (ERAV) and P2A from Porcine teschovirus-1 (PTV1).

In another aspect, the present invention provides a composition comprising at least one vector, such as a viral vector, wherein the composition comprises one vector as defined above; or said composition comprises at least two vectors, wherein one of the vectors comprises the nucleic acid molecule comprising a nucleotide sequence encoding an aCAR as defined above and another vector comprises the nucleic acid molecule comprising a nucleotide sequence encoding a homodimeric IL-10 as defined above.

The type of Treg cell selected is of importance for successful clinical implementation. Tr1 cells are a subset of CD4(+) FoxP3(+/−) Tregs which are induced in the periphery in a TCR- and antigen-specific manner upon chronic exposure to antigen on dendritic cells in the presence of IL-10 (26, 27). These cells are characterized by a non-proliferative (anergic) state, high production of IL-10 and TGF-β but only minimally of IL-2 and none of IL-4 or IL-17 and the ability to suppress Teffs in a cell-to-cell contact-independent manner. A recent study demonstrated that the enforced expression of IL-10 in human CD4 T cells, accomplished by lentiviral transduction, was sufficient for endowing these cells with a stable Tr1 phenotype in an autocrine fashion (1). This study also showed that exposure of these cells to IL-2 could temporarily reverse the anergic state of these IL-10-induced Tr1 cells. Importantly, two cell surface markers, CD49b and LAG-3, have been identified, which are stably and selectively co-expressed on human (and mouse) Tr1 cells and allow their isolation and flow cytometry analysis for purity of the cell population (28).

In the present invention we use a gene encoding a membrane-anchored derivative of IL-10 (mem-IL-10). This membrane IL-10 construct serves as an IL-10-driven safe lock guaranteeing permanent preservation of the Tr1 phenotype, while avoiding IL-10 secretion in the absence of antigenic stimulation (WO 2019/180724). Safety wise, as IL-10 does not signal T cell proliferation, the autonomous activation of the IL-10 signaling pathway is not associated with risk of uncontrolled cell growth.

Thus, in a further aspect, the present invention provides a mammalian regulatory T cell (Treg) comprising any one of the nucleic acid molecules as defined above, or the vector, such as a lentiviral vector and a retroviral vector optionally integrated into the genome of the cell, as defined above.

In certain embodiments, the mammalian Treg expresses on its surface an aCAR according to any one of the above embodiments, and optionally the mammalian Treg further expresses on its surface a homodimeric IL-10 that is linked to a transmembrane-intracellular stretch, optionally through a flexible hinge according to any one of the above embodiments.

In particular embodiments, the extracellular domain of the aCAR expressed on the mammalian cell is an scFv specifically binding PGN or a TLR-binding domain, such as a TLR2-binding domain.

The present invention further contemplates nucleotide sequences and vectors encoding, compositions comprising, and Tregs expressing more than one aCAR having various TLR-binding domains. For example, expression of an aCAR with a TLR2-binding domain and another aCAR with a TLR1- or TLR6-binding-domain facilitates formation of heterodimers of the TLR2-aCAR with the TLR1- or TLR6-aCAR, thereby extending the ligand repertoire. These aCARs have preferably a TIR-Zeta intracellular domain.

In particular, the mammalian Treg expressing the aCAR of the present invention also expresses on its surface homodimeric IL-10 that is linked to a transmembrane-intracellular stretch, optionally through a flexible hinge.

In certain embodiments, the mammalian Treg has a stable Tr1 phenotype exhibiting the cell-surface markers CD49b and LAG-3. In particular, Tregs that express membrane-bound homodimeric IL-10 as defined herein have a stable Tr1 phenotype exhibiting the cell-surface markers CD49b and LAG-3. Tregs that express only the CAR of the present invention, and not the membrane-bound homodimeric IL-10 as defined herein, tend to have a phenotype of ‘conventional Tregs’ that is that is useful for the purpose of the present invention but less stable and can be modified.

In certain embodiments, the mammalian Treg is a human Treg.

In certain embodiments, the mammalian Treg is an allogeneic or autologous Treg.

In still an additional aspect, the present invention provides a method of preparing allogeneic or autologous Tregs, the method comprising contacting CD4 T cells with the nucleic acid molecule comprising a nucleotide sequence encoding an aCAR according to any one of the above embodiments alone or in combination with a nucleotide sequence encoding a homodimeric IL-10 according to any one of the above embodiments, a vector comprising it, or a composition according to any one of the above embodiments, thereby preparing allogeneic or autologous Tregs expressing on their surface aCARs with or without mem-IL-10. As noted above, Tregs prepared by the method of the invention that express membrane-bound homodimeric IL-10 as defined herein have a stable Tr1 phenotype exhibiting the cell-surface markers CD49b and LAG-3. Tregs that express only the CAR of the present invention, and not the membrane-bound homodimeric IL-10 as defined herein, tend to have a phenotype of ‘conventional Tregs’ that is useful for the purpose of the present invention but less stable and can be modified.

Methods for isolating and preparing T cells, such as CD4 T cells, are well known in the art (1) and often rely on commercial kits and protocols from leading companies in this field:

1. ThermoFisher Scientific: Isolation of Untouched Human CD4+ T Cells from Peripheral Blood Mononuclear Cells (PBMC):

https://www.thermofisher.com/il/en/home/references/protocols/proteins-expression-isolation-and-analysis/cell-separation-methods/human-cell-separation-protocols/isolation-of-untouched-human-cd4-t-cells.html;

2. Miltenyi Biotec: CD4+ T Cell Isolation Kit, human;

https://www.miltenyibiotec.com/CA-en/products/macs-cell-separation/cell-separation-reagents/microbeads-and-isolation-kits/t-cells/cd4-t-cell-isolation-kit-human.html

3. STEMCELL Technologies: EasySep™ Human CD4+ T Cell Isolation Kit;

https://www.stemcell.com/easysep-human-cd4-t-cell-isolation-kit.html

4. BD Biosciences: Human Naive CD4 T Cell Enrichment Set

https://www.bdbiosciences.com/eu/reagents/research/magnetic-cell-separation/human-cell-separation-reagents/human-naive-cd4-t-cell-enrichment-set—dm/p/558521

The immune cells may be transfected with the appropriate nucleic acid molecule described herein by e.g. RNA transfection or by incorporation in a plasmid fit for replication and/or transcription in a eukaryotic cell or a vector, such as a viral vector described above. In certain embodiments, the vector is selected from a retroviral or lentiviral vector.

Combinations of retroviral vector and an appropriate packaging line can also be used, where the capsid proteins will be functional for infecting human cells. Several amphotropic virus-producing cell lines are known, including PA12 (29), PA317 (30); and CRIP (31). Alternatively, non-amphotropic particles can be used, such as, particles pseudotyped with VSVG, RD 114 or GAL V envelope. Cells can further be transduced by direct co-culture with producer cells, e.g., by the method of Bregni et al. (32), or culturing with viral supernatant alone or concentrated vector stocks, e.g., by the method of Xu, et al. (33) and Hughes, et al. (34).

The methods for creating recombinant retroviral and lentiviral vectors and using them for transducing T cells are usually performed by means of commercial kits including packaging cells, plasmids and transfection reagents, which are offered by many companies, including Invitrogen®, Sigma®, Clontech®, Cell Biolabs®, SBI®, Genecopoeia® and many others. The methods are thus performed along with the guidelines supplied with the commercial kits.

In short, according to a non-limiting example taught by the γ-Retrovirus Guide on the web site of Addgene, the following components are needed: (a) γ-Retroviral transfer plasmid encoding a transgene of interest: The transgene sequence is flanked by long terminal repeat (LTR) sequences, which facilitate integration of the transfer plasmid sequences into the host genome. Typically it is the sequences between and including the LTRs that is integrated into the host genome upon viral transduction; (b) Packaging genes (viral Gag-Pol): Gag is a structural precursor protein, and Pol is a polymerase; and (c) Envelope gene (may be pseudotyped to alter infectivity).

As a non-limiting example, the three components described above (envelope, packaging, and transfer) are supplied by three types of plasmids, which are cotransfected into a 293T packaging cell line. This system provides the greatest flexibility to pseudotype γ-retrovirus using different envelopes to modify tropism. Briefly, different envelope plasmids can direct the production of virus with various tropisms. A detailed non-limiting example of methods for preparation of recombinant retroviral stock and retroviral transduction of human CD4 T cells is found below in the Examples section.

In another aspect, the present invention provides a method of treating or preventing a disease, disorder or condition in a subject, comprising administering to said subject the mammalian Treg expressing on its surface an aCAR alone or in combination with a homodimeric IL-10 according to any one of the above embodiments, wherein said disease, disorder or condition is manifested in excessive activity of the immune system, such as an autoimmune disease, allergy, asthma, and organ and bone marrow transplantation.

In a similar aspect, the present invention provides the mammalian Treg expressing on its surface an aCAR alone or in combination with a homodimeric IL-10 according to any one of the above embodiments, for use in treating or preventing a disease, disorder or condition in a subject, wherein said disease, disorder or condition is manifested in excessive activity of the immune system, such as an autoimmune disease, allergy, asthma, and organ and bone marrow transplantation.

In a similar aspect, the present invention provides use of the mammalian Treg expressing on its surface an aCAR alone or in combination with a homodimeric IL-10 according to any one of the above embodiments, for use in the manufacture of a medicament for the treatment or prevention of a disease, disorder or condition in a subject, wherein said disease, disorder or condition is manifested in excessive activity of the immune system, such as an autoimmune disease, allergy, asthma, and organ and bone marrow transplantation.

The specific diseases defined as autoimmune diseases are well known in the art; for example, as disclosed in The Encyclopedia of Autoimmune Diseases, Dana K. Cassell, Noel R. Rose, Infobase Publishing, 14 May 2014, incorporated by reference as if fully disclosed herein.

The following are non-limiting examples of autoimmune and inflammatory diseases causing or associated with disease of the gut:

Systemic autoimmune diseases include collagen vascular diseases, the systemic vasculitides, Wegener granulomatosis, and Churg-Strauss syndrome. These disorders can involve any part of the gastrointestinal tract, hepatobiliary system and pancreas. They can cause a variety of gastrointestinal manifestations that are influenced by the pathophysiologic characteristics of the underlying disease process. There is a wide variation of gastrointestinal manifestations from these autoimmune disorders including, but not limited to: oral ulcers, dysphagia, gastroesophageal reflux disease, abdominal pain, constipation, diarrhea, fecal incontinence, pseudo-obstruction, perforation and gastrointestinal bleeding.

Systemic lupus erythematosus (SLE) is an autoimmune disease of unknown pathogenesis, characterized at histologic examination by deposition of autoantibodies and immune complexes that damage tissues and cells. The presentation is usually systemic and includes fatigue, malaise, anorexia, fever, and weight loss. The disease predominantly affects women (F:M, 10:1) aged 20-50 years. Gastrointestinal manifestations of SLE are common. GI symptoms are common in patients with SLE and can be due to primary gastrointestinal disorders, complications of therapy or SLE itself. Any part of the gastrointestinal tract may become involved in SLE.

Rheumatoid arthritis is an autoimmune disease of unknown pathogenesis that affects 1% of the population, with a 3:1 predilection for women between the ages of 20 and 50 years. The classic clinical manifestation is chronic symmetric polyarthritis due to a persistent inflammatory synovitis. Gastrointestinal manifestations are common.

Sjögren syndrome is a common autoimmune disease evidenced by broad organ-specific and systemic manifestations. B-cell activation is a consistent finding in patients with Sjögren syndrome, and B and T cells invade and destroy target organs. Sjögren syndrome usually affects women (F:M, 9:1) in the fourth and fifth decades of life. Although Sjögren syndrome affects approximately 2% of the adult population, it remains undiagnosed in more than half. Consequently, the interval between the onset of Sjögren syndrome and its diagnosis is frequently long-10 years, on average, according to one estimate. Patients with Sjögren syndrome may have involvement of their entire gastrointestinal tract.

Behçet's disease is a widespread vasculitis of unknown origin occurring in young patients, but people of all ages can develop this disease. Behçet's disease is an autoimmune disease that results from damage to blood vessels throughout the body, particularly veins. The exact cause of Behçet's disease is unknown. Most symptoms of the disease are caused by vasculitis. It was first defined as association of uveitis with oral and genital ulcers. However, now, the clinical spectrum also includes vascular, neurological, articular, renal and gastrointestinal manifestations. Gastrointestinal Behçet's disease shows a wide rage of sites of involvement and types of lesions.

Progressive systemic sclerosis (scleroderma) is a connective-tissue disease of unknown pathogenesis that affects 30- to 50-year-old women four times as often as it affects men. This type of sclerosis is characterized by overproduction of collagen, which leads to fibrosis of visceral organs. The overproduction of collagen is thought to result from an autoimmune dysfunction, in which the immune system would start to attack the kinetochore of the chromosomes. This would lead to genetic malformation of nearby genes. Any part of the gastrointestinal tract can be involved in scleroderma (Cojocaru M, Cojocaru I M, Silosi I, Vrabie C D. Gastrointestinal manifestations in systemic autoimmune diseases. Maedica (Buchar). 2011; 6(1):45-51.).

Inflammatory bowel disease (IBD) is a group of inflammatory conditions of the colon and small intestine. Crohn's disease and ulcerative colitis are the principal types of inflammatory bowel disease. Crohn's disease affects the small intestine and large intestine, as well as the mouth, esophagus, stomach and the anus, whereas ulcerative colitis primarily affects the colon and the rectum. IBD is a complex disease which arises as a result of the interaction of environmental and genetic factors leading to immunological responses and inflammation in the intestine.

Coeliac disease or celiac disease is a long-term immune disorder that primarily affects the small intestine. Classic symptoms include gastrointestinal problems such as chronic diarrhoea, abdominal distention, malabsorption, loss of appetite and among children failure to grow normally.

In certain embodiments, the autoimmune disease is selected from an inflammatory bowel disease, such as Crohn's disease and ulcerative colitis; celiac disease; type 1 diabetes; rheumatoid arthritis; systemic lupus erythematosus; Sjögren's syndrome; Behçet's disease; scleroderma; collagen vascular diseases; systemic vasculitides, Wegener granulomatosis; Churg-Strauss syndrome; psoriasis; psoriatic arthritis; multiple sclerosis; Addison's disease; Graves' disease; Hashimoto' s thyroiditis; myasthenia gravis; vasculitis; pernicious anemia; and atherosclerosis.

In certain embodiments, the autoimmune disease is selected from an inflammatory bowel disease, such as Crohn's disease and ulcerative colitis; type 1 diabetes; and celiac disease.

In certain embodiments, the autoimmune disease is an inflammatory bowel disease.

In certain embodiments, the subject is human and said mammalian Treg is human.

In some embodiments, Treg is an allogeneic Treg.

The Tregs used in the methods for treating diseases as defined above may be contacted with retinoic acid prior to administration to the subject in order to equip the reprogrammed Tr1 cells with gut homing capacity and to sustain Treg stability and function in the presence of IL-6 in an inflammatory environment.

Definitions:

The term “nucleic acid molecule” as used herein refers to a DNA or RNA molecule.

The term “extracellular domain” as used herein with reference to a protein means a region of the protein, which when expressed normally in a cell is located outside of the cell.

The terms “specific binding”, “specifically binding” or “capable of specifically binding” as used herein in the context of an extracellular binding-domain, such as an scFv, that specifically binds to an antigen or epitope, refers to the relative binding of the scFv to the intended ligand or antigen relative to the relative binding of the scFv to a different irrelevant antigen or epitope. Since this depends on the avidity (number of CAR copies on the T cell, number of antigen molecules on the surface of target cells and the affinity of the specific CARs used, a functional definition would be that the specific scFv would provide a significant signal in an ELISA against the intended antigen or epitope to which it is specific or cells transfected with a CAR displaying the scFv would be clearly labeled with the intended antigen or epitope in a FACS assay, while the same assays using a different irrelevant antigen or epitope would not give any detectable signal.

Selective binding includes binding properties such as, e.g., binding affinity, binding specificity, and binding avidity. Binding affinity refers to the length of time the binding-domain resides at its epitope binding site, and can be viewed as the strength with which a binding-domain binds its epitope. Binding affinity can be described as a binding-domain's equilibrium dissociation constant (KD), which is defined as the ratio Kd/Ka at equilibrium. Where Ka is the binding-domain's association rate constant and kd is the binding-domain's dissociation rate constant. Binding affinity is determined by both the association and the dissociation and alone neither high association or low dissociation can ensure high affinity. The association rate constant (Ka), or onrate constant (Kon), measures the number of binding events per unit time, or the propensity of the antibody and the antigen to associate reversibly into its antibody-antigen complex. The association rate constant is expressed in M-1 s-1, and is symbolized as follows: [Ab]×[Ag]×Kon. The larger the association rate constant, the more rapidly the antibody binds to its antigen, or the higher the binding affinity between antibody and antigen. The dissociation rate constant (Kd), or off-rate constant (Koff), measures the number of dissociation events per unit time propensity of an binding-domain-antigen complex to separate (dissociate) reversibly into its component molecules, namely the binding-domain and the antigen. The dissociation rate constant is expressed in s-1, and is symbolized as follows: [Ab+Ag]×Koff. The smaller the dissociation rate constant, the more tightly bound the antibody is to its antigen, or the higher the binding affinity between antibody and antigen. The equilibrium dissociation constant (KD) measures the rate at which new binding-domain-antigen complexes formed equals the rate at which binding-domain-antigen complexes dissociate at equilibrium. The equilibrium dissociation constant is expressed in M, and in the case of antibodies is defined as Koff/Kon=[Ab]×[Ag]/[Ab+Ag], where [Ab] is the molar concentration of the antibody, [Ag] is the molar concentration of the antigen, and [Ab+Ag] is the of molar concentration of the antibody-antigen complex, where all concentrations are of such components when the system is at equilibrium. The smaller the equilibrium dissociation constant, the more tightly bound the antibody is to its antigen, or the higher the binding affinity between antibody and antigen.

The binding specificity of a binding-domain or an aCAR comprising it as disclosed herein may also be characterized as a ratio that such a binding-domain/aCAR can discriminate its epitope relative to an irrelevant epitope. For example, a binding-domain/aCAR disclosed herein may have a binding specificity ratio for its epitope relative to an irrelevant epitope of, e.g., at least 2:1, at least 3:1, at least 4:1, at least 5:1, at least 64:1, at least 7:1, at least 8:1, at least 9:1, at least 10:1, at least 15:1, at least 20:1, at least 25:1, at least 30:1, at least 35:1, or at least 40:1.

It should be clear that a binding-domain of an aCAR described herein as specifically binding an antigen or epitope is meant to be capable of specifically binding the antigen or epitope and is not necessarily bound to it at any given time.

ScFvs are derived from monoclonal antibodies, a substantially homogeneous population of antibody molecules that contain only one species of antibody capable of binding a particular antigen i.e., the individual antibodies comprising the population are identical except for possible naturally occurring mutations that may be present in minor amounts. By definition, a monoclonal antibody binds to a single epitope or antigenic site and is therefore defined by its antigen structure. ScFv are commonly used as the binding domain in CARs.

Methods for cloning and producing scFv using known sequences encoding for monoclonal antibodies, as well as incorporating scFv sequences into the framework of a CAR, are well known in the art. For example, a sequence encoding for a scFv specific to a certain antigen, may be cloned upstream (i.e., to N-terminus) of the stalk-transmembrane-intracellular domains as described in the literature, such (21, 35, 44-50, 36-43).

The term “treating” as used herein refers to means of obtaining a desired physiological effect. The effect may be therapeutic in terms of partially or completely curing a disease and/or symptoms attributed to the disease. The term refers to inhibiting the disease, i.e. arresting its development; or ameliorating the disease, i.e. causing regression of the disease.

As used herein, the terms “subject” or “individual” or “animal” or “patient” or “mammal,” refers to any subject, particularly a mammalian subject, for whom diagnosis, prognosis, or therapy is desired, for example, a human.

Pharmaceutical compositions for use in accordance with the present invention may be formulated in conventional manner using one or more physiologically acceptable carriers or excipients. The carrier(s) must be “acceptable” in the sense of being compatible with the other ingredients of the composition and not deleterious to the recipient thereof.

The following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local.

The term “carrier” refers to a diluent, adjuvant, excipient, or vehicle with which the active agent is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

The compositions may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

The term “peripheral blood mononuclear cell (PBMC)” as used herein refers to any blood cell having a round nucleus, such as a lymphocyte, a monocyte or a macrophage. Methods for isolating PBMCs from blood are readily apparent to those skilled in the art. A non-limiting example is the extraction of these cells from whole blood using ficoll, a hydrophilic polysaccharide that separates layers of blood, with monocytes and lymphocytes forming a buffy coat under a layer of plasma or by leukapheresis, the preparation of leukocyte concentrates with the return of red cells and leukocyte-poor plasma to the donor.

For purposes of clarity, and in no way limiting the scope of the teachings, unless otherwise indicated, all numbers expressing quantities, percentages or proportions, and other numerical values recited herein, should be interpreted as being preceded in all instances by the term “about.” Accordingly, the numerical parameters recited in the present specification are approximations that may vary depending on the desired outcome. For example, each numerical parameter may be construed in light of the number of reported significant digits and by applying ordinary rounding techniques.

The term “about” as used herein means that values of 10% or less above or below the indicated values are also included.

### EXAMPLES

### Example 1. Two IL-10 Monomers Linked Together in Tandem by a Flexible Linker and Linked to a Transmembrane-Intracellular Stretch via a Short Hinge Region

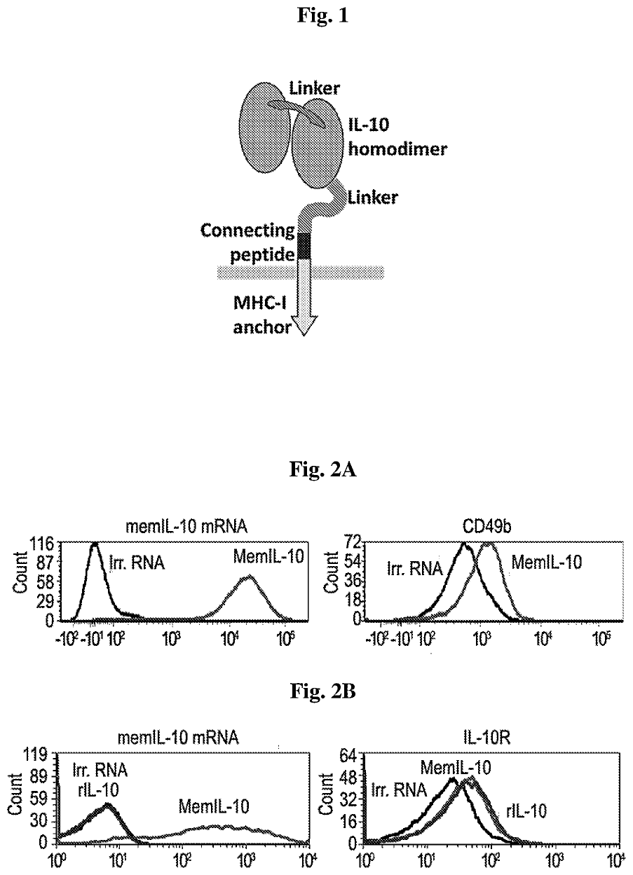

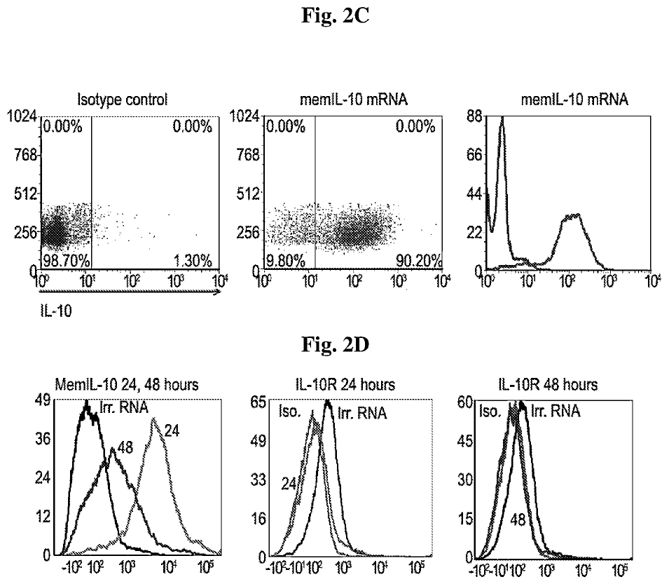

In the specific construct used here, two IL-10 monomers were linked together in tandem by a flexible linker of the sequence GSTSGSGKPGSGEGSTKG to create a homodimer, which was then linked to the transmembrane-intracellular stretch derived from the HLA-A2 heavy chain by a flexible hinge regions having a 21 amino acid spacer comprising the flexible linker Gly₄Ser(Gly₃Ser)₂ and an additional 8 amino acid bridge of the sequence SSQPTIPI derived from the membrane-proximal part of the connecting peptide of HLA-A2 (FIG. 1). Surface expression of memIL-10 and IL-10R on human and mouse CD4 T cells was then confirmed (FIG. 2).

Elevation of the CD49b integrin could be observed in (A) and upregulation of IL-10 receptor (IL-10R) was similar to that induced by recombinant IL-10 (rIL-10, (B)). Mouse memIL-10 was clearly expressed 48 hours post-transfection (D, left) and, as expected, memIL-10 blocked the binding of the anti-mouse IL-10R mAb we used, suggesting binding in-cis (51).

### Example 2. Two IL-10 Monomers Linked Together in Tandem by a Flexible Linker and Linked to a Transmembrane-Intracellular Stretch via a Long Hinge Region or the IL-10R β Chain

Our original memIL-10 constructs, both human and mouse, incorporated a hinge comprising a flexible linker of 21 amino acids (in addition to an 8 amino acid-long rigid spacer, now termed SmemIL-10 (S for short linker, see below).

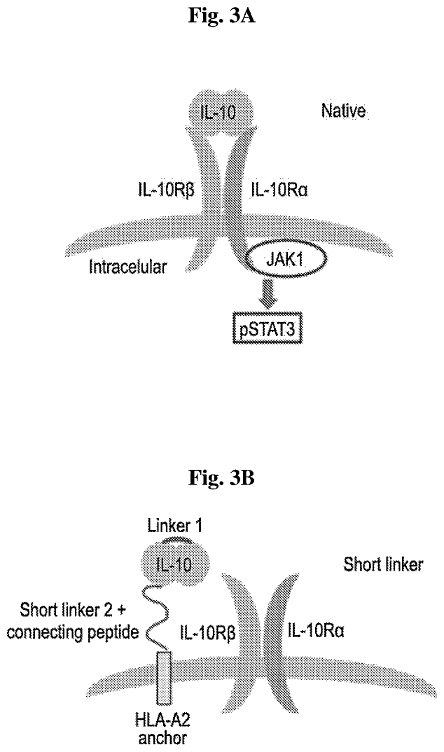

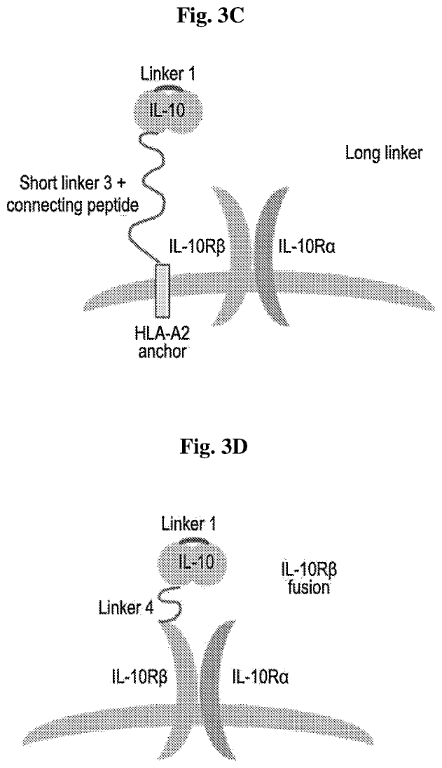

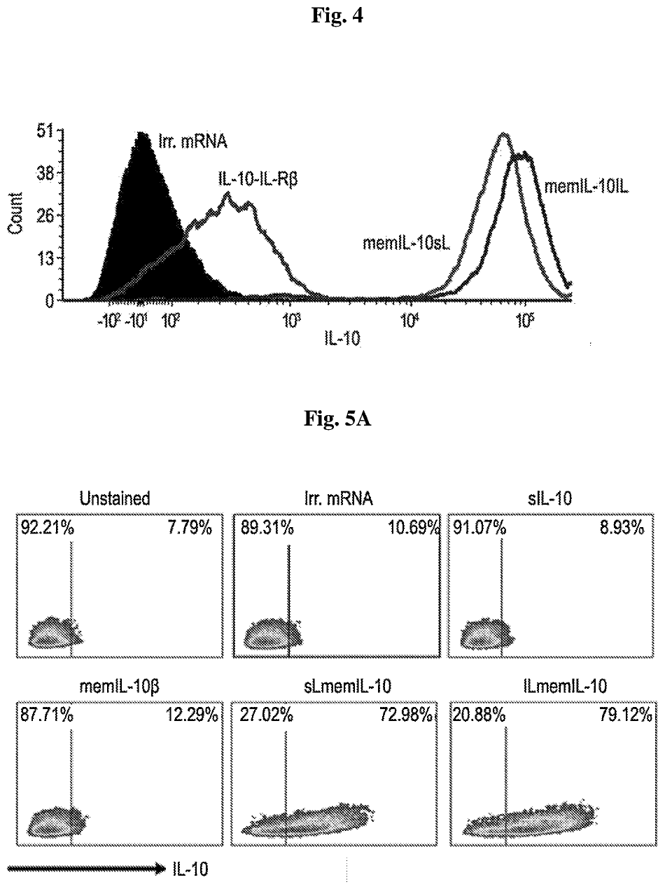

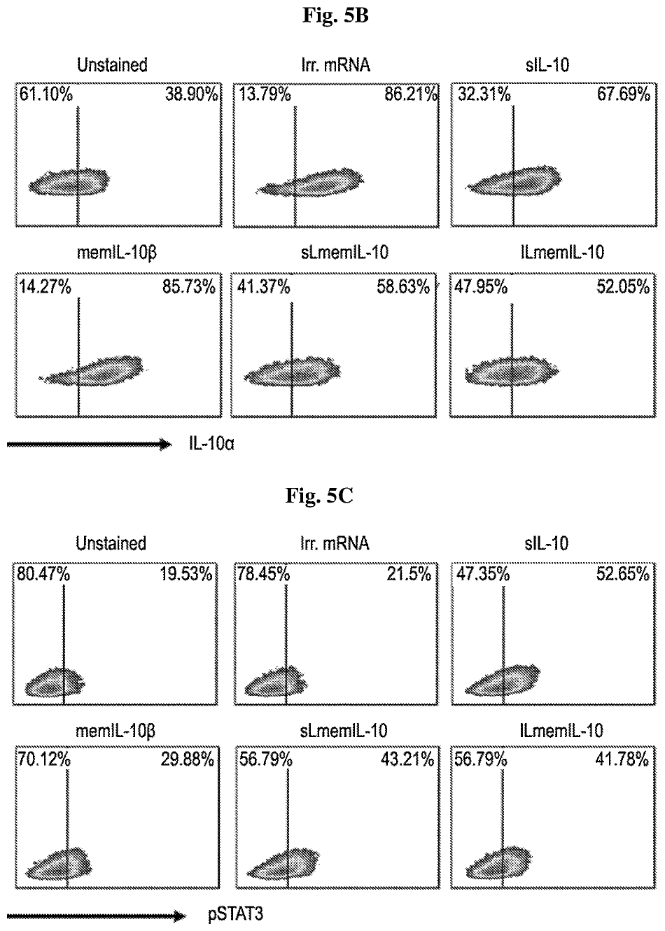

In an attempt to optimize our memIL-10 we have engineered and cloned two new versions of this membrane cytokine: In one, cloned first, we provided memIL-10 with a longer linker peptide (of 30 amino acids, termed LmemIL-10 for long) to facilitate optimal engagement with IL-10R (FIG. 3, lower left). To create another derivative we fused our dimeric IL-10 to the N-terminus of the IL-10R β chain as a new scaffold designed to endow it with direct access to the IL-10 binding site located on the IL-10R α chain, designated memIL-10RB (FIG. 3, lower right). Indeed, FIG. 4 confirms surface expression of the three products in human Jurkat cells. Of note, it is expected that the level of surface expression of the memIL-10RB fusion protein depend on the availability of IL-10Rα chain. To evaluate expression and function of the three different memIL-10 configurations mouse CD4 T cells were transfected with mRNA encoding the three constructs and assayed for surface expression (FIG. 5A), downregulation of surface IL-10R (FIG. 5B) and spontaneous phosphorylation of STAT3 (FIG. 5C). Indeed, in agreement with the results obtained in Jurkat cells, the constructs harboring the short and long linkers are expressed at much higher levels than memILL-10Rβ and exhibit superior function, as evident from the greater reduction in surface IL-10R and the stronger induction of pSTAT3. As the short linker construct (sLmemlL-10) was superior to the long linker one (1LmemIL-10) in its ability to induce pSTAT3 also in repeated experiments (not shown) it was selected for further experiments.

### Example 3. Expression and Characterization of memIL-10 in Retrovirally Transduced Mouse CD4 T Cells

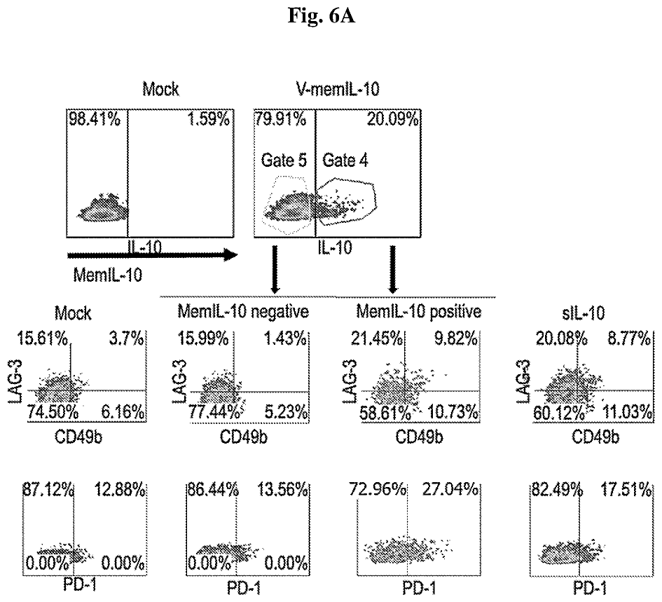

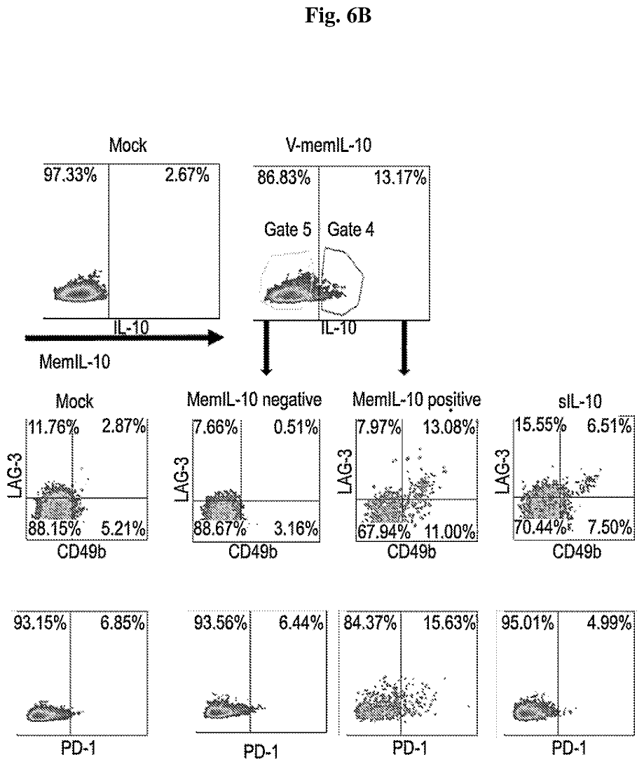

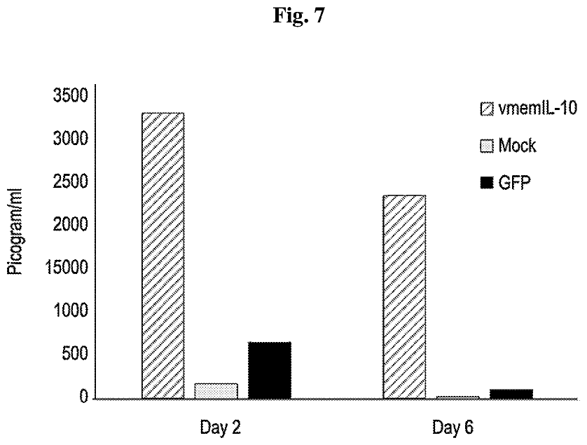

To test expression and function of memIL-10 in retrovirally transduced T cells we first used splenic CD4 T cells purified with magnetic beads from C57BL/6 (B6) mice. As a negative control for memIL-10 transduced cells we used mock-transduced cells (Mock). Soluble IL-10 (sIL-10) was used in these experiments as a positive control. FIG. 6 shows the results of a flow cytometry analysis of transduced cells vs. non-transduced ones which grew in the same culture and mock-transduced cells for the expression of the three Tr1-associated markers LAG-3, CD49b and PD-1 48 hours and 6 days post-transfection. Clear elevation of the 3 markers could indeed be observed already at day 2 which also persisted at day 6, pointing the expected phenotype. The ability of the transduced T cells to secrete IL-10 upon TCR-mediated activation confirmed the acquisition of Tr1-like functional properties (FIG. 7).

### Example 4. Prolonged Expression of memIL-10 and Phenotype Characterization

Prolonged expression of memIL-10 is achieved by retroviral transduction. For control non-Tr1 CD4 T cells, CD4 T cells are transduced with the EGFP gene as a marker. We first attempt to establish an effective protocol (examining the need for irradiated APCs, TCR stimulation, cytokines and other culture conditions, following detailed guidelines provided in (52, 53) for mouse and (1) for human CD4 T cells) for differentiating CD4 T cells of NOD and C57BL/6 (B6) mice, which are relevant to several in-vivo disease models, into Tr1 cells. To this end we use flow cytometry analysis to correlate acquisition of LAG-3 and CD49b with memIL-10 expression. Additional phenotypic analyses (all in comparison with EGFP+non-Tr1 cells) determine rate of in-vitro expansion, status of differentiation (CD45RO+, CD45RA−, CD62L), level of activation markers (CD40L, CD40, CD25, FOXP3, CD161, and CD137) and markers associated with IL-10 (PD-1, ICOS-L, ICOS and IL-10R). The function of memIL-10-induced Tr1 cells is first evaluated via the pattern of cytokines they secrete in response to TCR-mediated activation, including IL-10, TGF-β, IFN-γ, IL-2, IL-4, IL-5 and TNF-α. To assess the anergic state we analyze proliferative capacity in the presence of anti-CD3 and anti-CD28 Abs and in the absence or presence of soluble IL-2 and IL-15, using a CFSE dilution assay.

### Example 5. Assessing Inhibitory Effect of Transduced Cells on T Effector Cells

To examine the ability of transduced cells to exert their inhibitory effect on neighboring Teff cells we design a coculture setting which allows us to selectively activate at will only one T cell population and not the other (obviously, anti-TCR/CD3 Abs would activate all T cells in the coculture). To this end we exploit two genes we have created, encoding the chimeric H-2Kb-CD3ζ (Kb-CD3ζ) and H-2Kd-CD3ζ (Kd-CD3ζ) MHC-I heavy chains. We have already shown that both genes selectively activate T cells following Ab-mediated cross-linking in magnitude that is comparable to TCR cross-linking. In the following series of functional experiments we employ these tools to mix mRNA-transfected Tr1 and Teff cells at different ratios for 3-4 days and use CFSE dilution and intracellular IFN-γ staining to assess the ability of activated Tr1 cells (vs. non-activated or RFP+ non-Tr1 cells) to suppress both proliferation and effector function of the activated Teffs.

### Example 6. Assessing in-vivo Persistence of IL-10-Transduced Cells and Suppressive Function in Mouse Models for Human Diseases

To evaluate in-vivo persistence of the IL-10-transduced NOD or B6 CD4 T cells in syngeneic wild-type mice and maintenance of their phenotype a protocol we recently established in our T1D experimental system (54) is used. Briefly, 10×10⁶ cells will be injected into the tail vain. Spleen and peripheral lymph nodes are harvested 1, 7 and 14 days post-injection and CD4+IL-10+LAG-3+CD49b+ T cells will be identified by flow cytometry (compared to background level of staining in non-injected mice).

The actual suppressive function of memIL-10-tarsduced T cells under physiological conditions in-vivo is then tested, employing mouse models for human diseases such as T1D or IBD.

### Example 7. Expression and Characterization of memIL-10 in Retrovirally Transduced Human CD4 T Cells

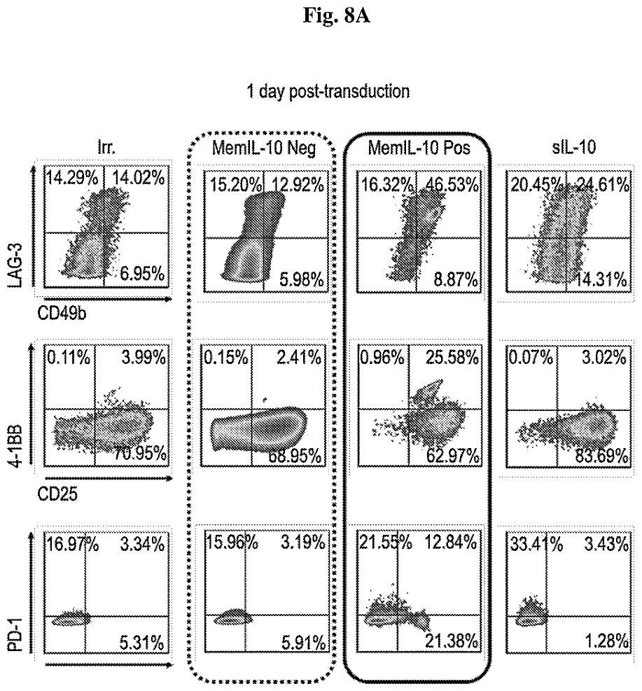

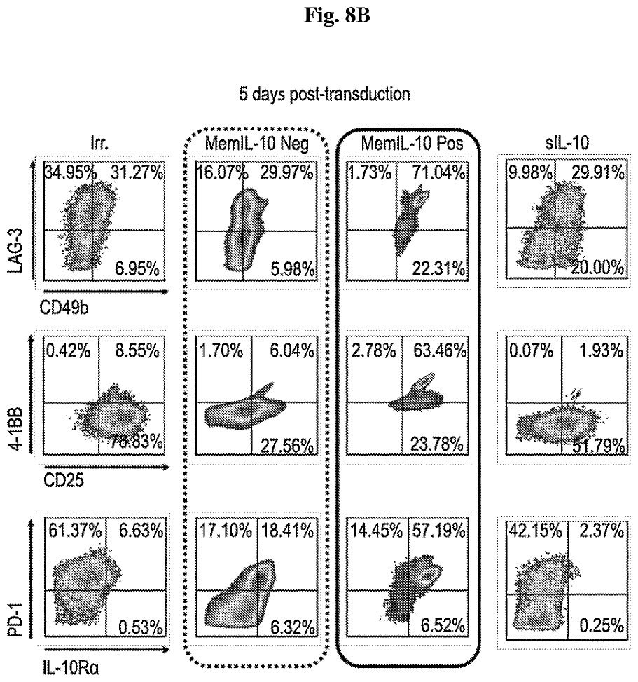

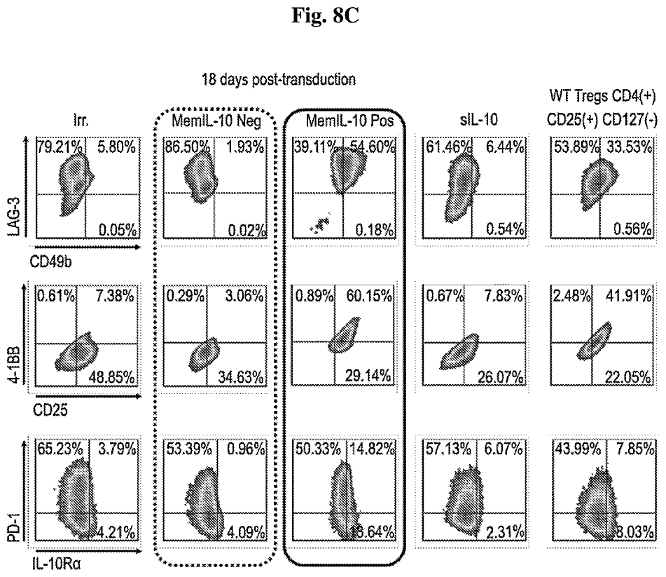

For assessing the phenotypic and functional outcome of retroviral transduction of human CD4 T cells we isolated CD4 T cells from blood samples obtained from healthy donors through the Blood Services Center of Magen David Adom, Israel. The first of two independent ex-vivo experiments is presented in FIG. 8. In this experiment cells have been kept in culture eighteen days post-transduction and phenotypic analyses for the markers LAG-3, CD49b, PD-1, 4-1BB, CD25 and IL-10Rα were performed by flow cytometry at days 1, 5 and 18 post-transduction. Our results confirm that all these cell surface markers that are associated with the expected Tr1 phenotype were significantly increased in memIL-10-expressing cells compared to memIL-10-negative cells that grew in the same culture dish for the entire period of the experiment.

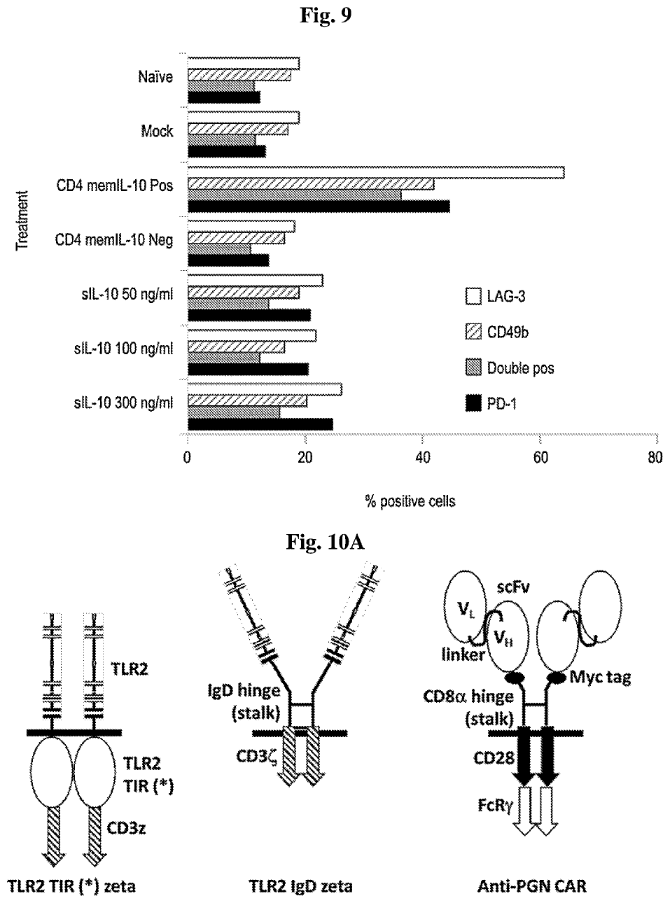

The second experiment was performed on a different blood sample and flow cytometry performed for LAG-3, CD49b and PD-1 (FIG. 9) are in line with the results obtained in the first experiment. From these two experiments it can be concluded that long-term expression of memIL- 10 in human CD4 T cells via retroviral transduction endows these cells with a TR-1-like phenotype.

### Example 8. Inflammatory Bowel Disease (IBD) Treatment Application

This invention offers a solution to the need in identifying suitable antigens for redirecting CAR-Tregs at IBD-associated antigens for restoring immune tolerance at the inflamed gut.

The approach is based on the following concepts:

Tregs are genetically redirected against a common gut antigen derived from either the commensal microflora or food, which can cross the intestinal epithelium.

As first choice, Treg retargeting is implemented via a chimeric antigen receptor (CAR) comprising the extracellular portion of TLR2, which naturally binds the common bacterial constituent peptidoglycan and additional intestinal microbial antigens. Alternatively, a conventional scFv-based CAR against PGN is generated.

The Tregs of choice are type 1 regulatory T cells (Tr1), which, following TCR-mediated engagement with antigen, can suppress inflammatory T cells in a cell-to-cell-independent manner, mostly through the secretion of high amount of IL-10 and TGF-β.

Since enforced expression of IL-10 in human CD4 T cells is sufficient to both induce and maintain a Tr1 phenotype, the expression of membrane-bound IL-10 serves as a new device for exploiting this property in an autocrine manner.

Two recently identified surface markers, CD49b and LAG-3, which are selectively and stably expressed on Tr1 cells, allow their purification and subsequent analysis for preservation of the Tr1 phenotype.

(optional, contingent upon the identification of a proper candidate antigen: an inhibitory CAR specific to a dietary antigen co-expressed in the same Tr1 cells serves as a unique means to temporarily shut-off the suppressive function of CAR-Tregs (e.g., in case of infection)).

Gut homing of redirected Tr1 cells can be enhanced by incubation with all-trans retinoic acid prior to infusion.

### Example 9. The Immunotargeting Device

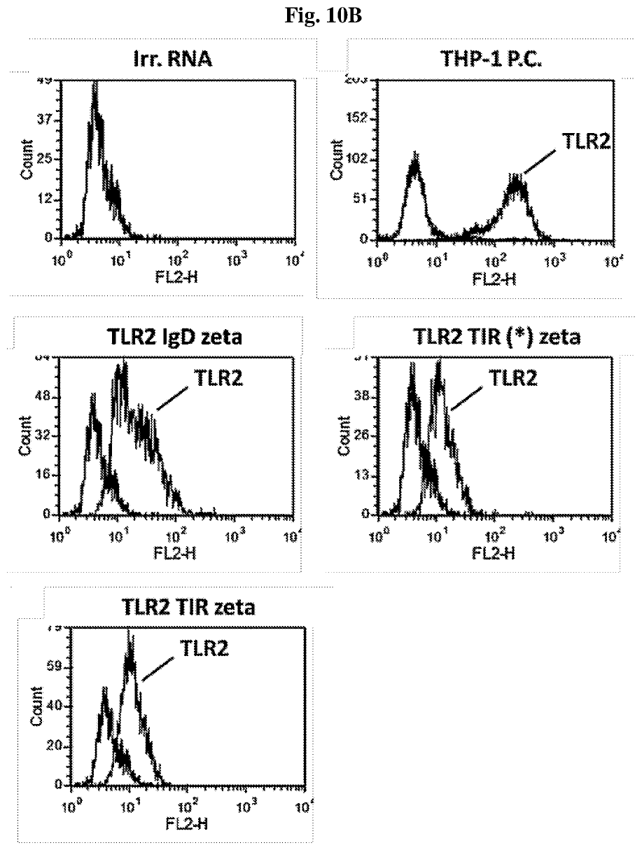

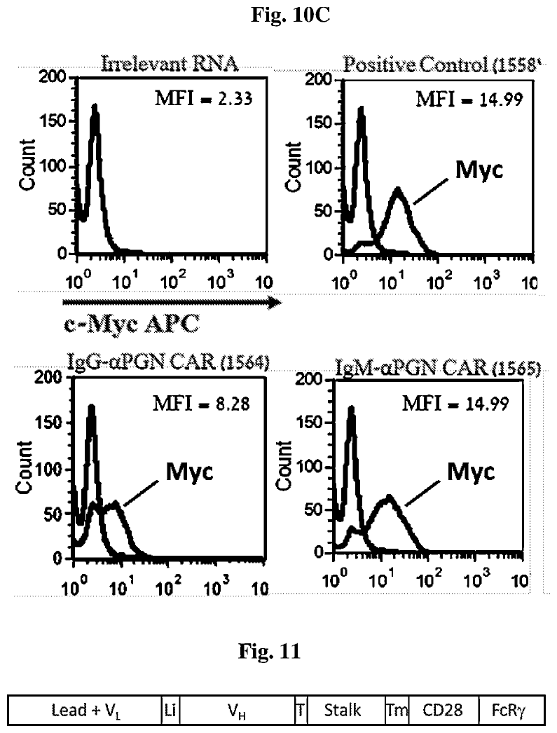

This example describes the genetic engineering of TLR2-based CARs for redirecting Tregs to PGN. TLR5-based CARs against flagellin or other TLR-CARs are constructed following the same guidelines. Two cloning strategies are illustrated in FIG. 10. The first (FIG. 10A, left) exploits full length TLR2. The T cell signaling moiety, in this case comprising CD3 is genetically engrafted onto the C-terminus of the TLR2 toll IL-1 receptor domain (TIR). Binding to PGN is expected to deliver two signals simultaneously, through TLR2 and CD3ζ, or through CD3ζ only when incorporating a well-studied Pro681His mutation in human TLR2 TIR (20), marked here as *. The second strategy engrafts TLR2 extracellular domain (ectodomain) onto a conventional hinge-CD3ζ CAR scaffold (FIG. 10A, middle), where the hinge region is derived from the human IgG or IgD heavy chain. FIG. 10A, right shows a ‘classical’ CAR based on an antibody single-chain Fv (scFv) fragment. In the context of the current invention this configuration can be exploited for the generation of e.g. an anti-PGN or anti-flagellin CAR using the scFv portion of an anti-PGN/flagellin mAb of choice.

To the best of our knowledge, TLR-based CARs have never been described. They can either allow the coupling of TLR recognition and signaling with T cell activation signaling (as in FIG. 10A, left, no *) or the TLR recognition (but no signaling) with T cell activation signaling (as in FIG. 10A, left, with * and FIG. 10A middle). TLR-mediated recognition by these TLR-CARs is expected to recapitulate the physiological recognition of multiple natural ligands by different TLRs.

### Example 10. Construction and Characterization of Anti-PGN aCARs.

Materials and Methods

Construction of scFv Gene Segments from two B Cell Hybridomas producing anti-PGN mAbs

To produce anti-PGN CARs we obtained from the ATCC two mouse B cell hybridomas producing mAbs specifically reactive with PGNs from a variety of gram− and gram+ bacteria. These are:

### 1. 3C11 (ATCC® HB-8511™), IgGl(κ)

https://www.atcc.org/Products/All/HB-8511.aspx#generalinformation

### 2. 3F6 (ATCC® HB-8512™), IgM(κ)

https://www.atcc.org/products/all/HB-8512.aspx#generalinformation

The full DNA sequences of the V_{H} and V_{L} genes of both these hybridomas has been determined (outsourcing, Hylabs, Rehovot, Israel) and served for cloning of their scFvs derivatives. These gene segments were then incorporated into a 2^{nd}-generation CAR backbone we have previously assembled in our lab (see FIG. 11: Lead, leader peptide; Li, linker; T, Myc Tag; hinge, derived from CD8α; Tm, CD28 transmembrane) For details of methods for preparing CARs, please see (21) (46) (47) (48) (49) (50) incorporated by reference as if fully disclosed herein.

The DNA Sequences of the anti-PGN CAR 1564 (3C11): scFV-CD28-γ

5′ untranslated sequence (SEQ ID NO: 2)-Leader peptide+V_{L} (SEQ ID NO: 4)-Linker (SEQ ID NO: 6)-V_{H} (SEQ ID NO: 8)-Myc tag (SEQ ID NO: 69)-CD8α hinge (SEQ ID NO: 10)-CD28 transmembrane & intracellular domains sequence (SEQ ID NO: 12)-FcRγ intracellular domain (SEQ ID NO: 14)-3′ untranslated sequence (SEQ ID NO: 15).

The Amino Acid Sequence of 1564 (3C11): scFV-CD28-γ, Protein

Leader peptide+V_{L} (SEQ ID NO: 3)-Linker (SEQ ID NO: 5)-V_{H} (SEQ ID NO: 7)-Myc tag (SEQ ID NO: 68)-CD8α hinge (SEQ ID NO: 9)-CD28 transmembrane & intracellular domains sequence (SEQ ID NO: 11)-FcRγ intracellular domain (SEQ ID NO: 13)

The DNA Sequence of 1565 (3F6): scFV-CD28-γ:

5′ untranslated sequence (SEQ ID NO: 2)-Leader peptide+V_{L} (SEQ ID NO: 17)-Linker (SEQ ID NO: 6)-V_{H} (SEQ ID NO: 19)-Myc tag (SEQ ID NO: 69)-CD8α hinge (SEQ ID NO: 10)-CD28 transmembrane & intracellular domains sequence (SEQ ID NO: 12)-FcRγ intracellular domain (SEQ ID NO: 14)-3′ untranslated sequence (SEQ ID NO: 15).

Amino Acid Sequence of 1565 (3F6): scFV-CD28-γ

Leader peptide+V_{L} (SEQ ID NO: 16)-Linker (SEQ ID NO: 5)-V_{H} (SEQ ID NO: 18)-Myc tag (SEQ ID NO: 68)-CD8α hinge (SEQ ID NO: 9)-CD28 transmembrane & intracellular domains sequence (SEQ ID NO: 11)-FcRγ intracellular domain (SEQ ID NO: 13)

Results:

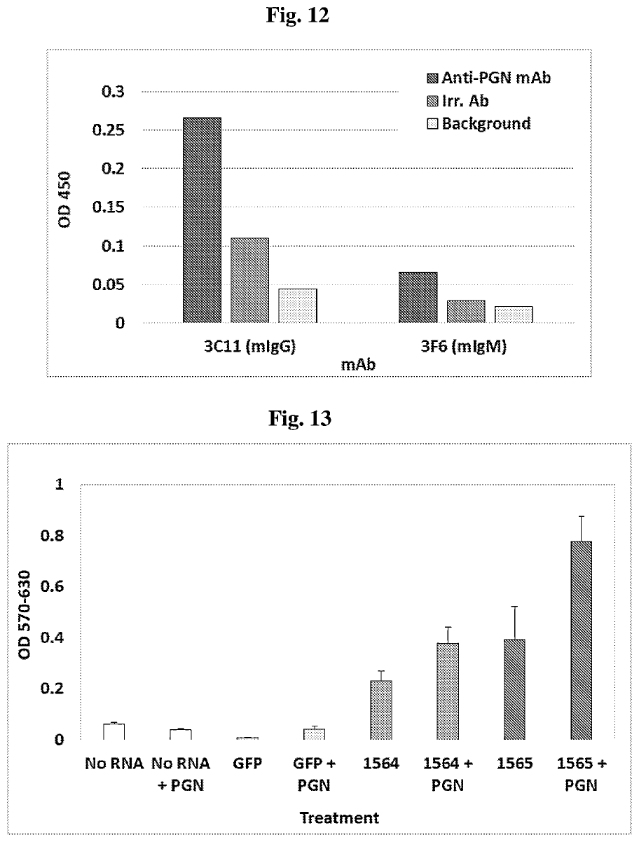

The two anti-PGN mAb, 3C11 (mouse IgG, purified from hybridoma) and 3F6 (mouse IgM, hybridoma supernatant) were assayed for binding PGN from S. aureus using an ‘Eppendorf ELISA’, and found to specifically bind PGN (FIG. 12).

Activation of anti-PGN CAR-T cells. B3Z T cells (T cell hybridoma expressing a TCR that specifically recognizes OVA(257-264) (SIINFEKL) in the context of H-2Kb) carrying the nuclear factor of activated T cells (NFAT)-LacZ reporter gene for T cell activation were transfected with mRNA encoding each of the two anti-PGN CARs (CAR-3C11 and CAR-3F6) or Green Fluorescent Protein (GFP) as a control. Cells were then incubated overnight in the presence or absence of PGN from S. aureus. Results are presented as OD of the colorimetric chlorophenol red-β-D-galactopyranoside (CPRG) assay for β-Gal activity (FIG. 13).

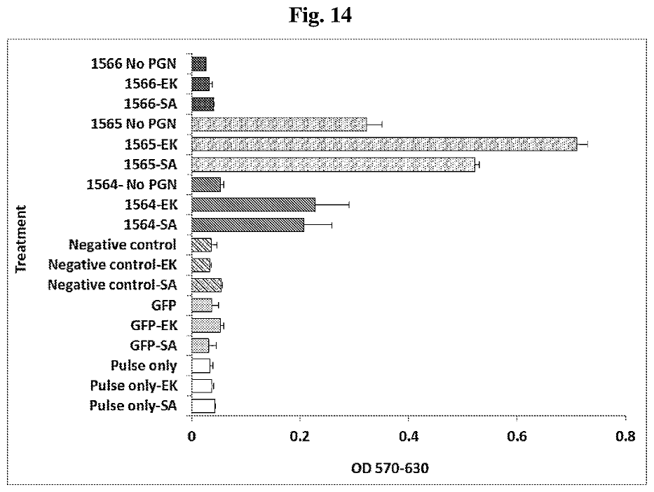

In the following experiment, the same B3Z reporter T cells were electroporated with mRNA encoding the two anti-PGN CARs and controls and, this time, cultured in the presence of PGN derived from both Gram-negative (E. coli) or Gram-positive (S. aureus) bacteria. 24 hours later cells were subjected to the colorimetric CPRG reporter assay for T cell activation (FIG. 14).

It is clear from FIGS. 13 and 14 that both anti-PGN CARs activate the T cells in a PGN-dependent manner. PGN from Gram-negative and Gram-positive bacteria were equally effective in activating the T cells.

### Example 11. Construction and Characterization of TLR2-aCARs

Construction of the TLR2-TIR(*)-zeta CARs

The general structure of the TLR2-TIR(*)-zeta CARs is depicted in FIG. 10A, left. Genes encoding two CARs of this series have been assembled, using modular restriction site-aided cloning. The gene for the TLR-2-TIR-Zeta CAR comprises the human TLR-2 cDNA, which encodes the leader peptide, the ectodomain, transmembrane and the wildtype TIR endodomain, followed by the full intracellular portion of human CD3ζ. The same components are included in the gene encoding the TLR-2-TIR(*)-Zeta CAR, except for the replacement of a C with A in the 681^{st} codon, which changes a Pro into His codon, producing the well-studied Pro681His mutation in human TLR2 TIR (20).

Construction of the TLR2-IgD-zeta CAR

Similarly to the TLR-2-TIR(*)-zeta CARs, the TLR-2-IgD-zeta CAR harbors the TLR2 ectodomain as the recognition moiety, which is engrafted on conventional 1^{st} generation CAR backbone comprising human IgD hinge and the transmembrane and intracellular portion of CD3-ζ (FIG. 10A, middle).

Expression of the TLR-2 aCARs Integrity and cell surface expression of the TLR-2-based CARs was confirmed by flow cytometry analysis following electroporation of in-vitro-transcribed mRNA encoding these CARs (FIG. 10B).

### Example 13. Capability of Anti-PGN aCARs Redirected Tr1 T Cells to Inhibit Anti-PGN Effector T Cells

In a series of two-party and three-party coculture experiments we examine the ability of the anti-PGN CAR or TLR2-CAR-transfected Tr1 cells (activated by PGN in culture and optionally transfected with a memIL-10 encoding vector) to suppress GFP-labeled activated Teffs as well as standby CD4 Teffs (expressing K^{d}-CD3ζ and activated by anti-K^{d} Abs) at different cell ratios. Readouts include intracellular staining for IFN-γ, IL-1, IL-6, TNF-α and TGF-β, gating on cells expressing the respective marker.

### Example 14. Gut Homing

The CAR expressing Tregs may be equipped with gut homing capacity by contacting them with Retinoic acid as described above.

Retroviral vectors encoding TLR2 or scFv anti-PGN-based CAR (our CD28-FCRγ signaling domain acts both in human and mouse T cells) and membrane IL-10 (human IL-10 binds and activates the mouse receptor) are assembled in two separate retroviral vectors or together in a bidirectional vector. Intact soluble IL-10 is also cloned as control (based on (1). Surface expression is validated. The ability of the TLR2 CAR to specifically redirect human T cells to PGN and of membrane IL-10 to trigger constitutive signaling is assessed (the latter with an IL-10 reporter gene we have already generated).

The TLR2/scFv anti-PGN-based CAR also serve for the generation of a readily available source for human and mouse PGN-specific Teff cells to be suppressed by gene-modified Tr1 cells.

In-vivo evaluation of this approach exploits the following rationale: Trinitrobenzenesulfonic acid (TNBS)- and oxazolone-induced colitis are two widely explored mouse model systems for IBD which employ these haptenating substances dissolved in ethanol via their intrarectal administration (55). These systems were formerly established in BALB/c mice and were utilized for the study of adoptively transferred trinitrophenyl (TNP)-redirected Tregs. These were derived from either transgenic mice expressing an anti-TNP CAR on a BALB/c background (56) or via retroviral transduction of CD4(+) CD25(+) Tregs isolated from wild-type BALB/c mice (57). Whereas TNP- redirected Tregs could suppress TNBS-induced colitis, they were ineffective against the oxazolone-driven disease and could only suppress this colitis when affected mice were also exposed to TNBS (56). This antigen-specific suppression puts forward the following conjecture which is addressed experimentally applying the protocols practiced in the Eshhar's lab: BALB/c-derived TLR2-CAR Tr1 cells are expected to suppress both TNBS- and oxazolone-induced colitis due to the ubiquitous presence of PGN in the gut, whereas TNP-CAR Tr1 cells would only suppress the TNBS-induced disease. Furthermore, following adoptive transfer to healthy BALB/c mice, PGN-redirected Tr1 cells would be constantly activated by antigen and, consequently, persist, whereas in the absence of antigen, their TNP-redirected counterparts are expected to be short-lived and disappear. (Note that these Tr1 cells are derived from the pool of CD4 Teff cells and not from natural Tregs which can still receive constant stimulus by cognate self-antigen through the endogenous TCR).

Accordingly, it is expected that PGN-specific but not TNP-specific Tr1 cells could provide protection from colitis induced by TNBS (and oxazolone) even if administered long before disease induction. Validation of this conjecture would provide strong support to the predicted stable Tr1 phenotype and long-term functionality of the reprogrammed T cells.

### REFERENCES

1. Andolfi, G., G. Fousteri, M. Rossetti, C. F. Magnani, T. Jofra, G. Locafaro, A. Bondanza, S. Gregori, and M.-G. Roncarolo. 2012. Enforced IL-10 expression confers type 1 regulatory T cell (Tr1) phenotype and function to human CD4+ T cells. Mol. Ther. 20: 1778-1790.

2. Abraham, C., and J. H. Cho. 2009. Inflammatory bowel disease. N Engl J Med 361: 2066-2078.

3. Maloy, K. J., and F. Powrie. 2011. Intestinal homeostasis and its breakdown in inflammatory bowel disease. Nature 474: 298-306.

4. Sakaguchi, S., F. Powrie, and R. M. Ransohoff. 2012. Re-establishing immunological self-tolerance in autoimmune disease. Nat. Med. 18: 54-58.

5. Himmel, M. E., Y. Yao, P. C. Orban, T. S. Steiner, and M. K. Levings. 2012. Regulatory T-cell therapy for inflammatory bowel disease: More questions than answers. Immunology 136: 115-122.

6. Desreumaux, P., A. Foussat, M. Allez, L. Beaugerie, X. Hébuterne, Y. Bouhnik, M. Nachury, V. Brun, H. Bastian, N. Belmonte, M. Ticchioni, A. Duchange, P. Morel-Mandrino, V. Neveu, N. Clerget-Chossat, M. Forte, and J.-F. Colombel. 2012. Safety and efficacy of antigen-specific regulatory T-cell therapy for patients with refractory Crohn's disease. Gastroenterology 143: 1207-1217.e2.

7. Mazmanian, S. K., C. H. Liu, A. O. Tzianabos, and D. L. Kasper. 2005. An immunomodulatory molecule of symbiotic bacteria directs maturation of the host immune system. Cell 122: 107-18.

8. Clarke, T. B., K. M. Davis, E. S. Lysenko, A. Y. Zhou, Y. Yu, and J. N. Weiser. 2010. Recognition of peptidoglycan from the microbiota by Nod1 enhances systemic innate immunity. Nat Med 16: 228-231.

9. Hiemstra, I. H., K. Vrijland, M. M. Hogenboom, G. Bouma, G. Kraal, and J. M. M. den Haan. 2015. Intestinal epithelial cell transported TLR2 ligand stimulates Ly6C⁺ monocyte differentiation in a G-CSF dependent manner. Immunobiology 220: 1255-65.

10. Girardin, S. E., I. G. Boneca, J. Viala, M. Chamaillard, A. Labigne, G. Thomas, D. J. Philpott, and P. J. Sansonetti. 2003. Nod2 is a general sensor of peptidoglycan through muramyl dipeptide (MDP) detection. J Biol Chem 278: 8869-8872.

11. Tanabe, T., M. Chamaillard, Y. Ogura, L. Zhu, S. Qiu, J. Masumoto, P. Ghosh, A. Moran, M. M. Predergast, G. Tromp, C. J. Williams, N. Inohara, and G. Nunez. 2004. Regulatory regions and critical residues of NOD2 involved in muramyl dipeptide recognition. Embo J 23: 1587-1597.

12. Iwaki, D., H. Mitsuzawa, S. Murakami, H. Sano, M. Konishi, T. Akino, and Y. Kuroki. 2002. The extracellular toll-like receptor 2 domain directly binds peptidoglycan derived from Staphylococcus aureus. J Biol Chem 277: 24315-24320.

13. Asong, J., M. A. Wolfert, K. K. Maiti, D. Miller, and G. J. Boons. 2009. Binding and Cellular Activation Studies Reveal That Toll-like Receptor 2 Can Differentially Recognize Peptidoglycan from Gram-positive and Gram-negative Bacteria. J Biol Chem 284: 8643-8653.

14. Levine, A. G., A. Arvey, W. Jin, and A. Y. Rudensky. 2014. Continuous requirement for the TCR in regulatory T cell function. Nat. Immunol. 15: 1070-1078.

15. Gross, G., T. Waks, and Z. Eshhar. 1989. Expression of immunoglobulin-T-cell receptor chimeric molecules as functional receptors with antibody-type specificity. Proc Natl Acad Sci USA 86: 10024-8.

16. Gross, G., and Z. Eshhar. 2016. Therapeutic Potential of T Cell Chimeric Antigen Receptors (CARs) in Cancer Treatment: Counteracting Off-Tumor Toxicities for Safe CAR T Cell Therapy. Annu. Rev. Pharmacol. Toxicol. 56: 59-83.

17. 2014. Antibodies: A Laboratory Manual, Second edition, (Edward A. Greenfield, ed). CSH Press.

18. Dotti, G., S. Gottschalk, B. Savoldo, and M. K. Brenner. 2014. Design and development of therapies using chimeric antigen receptor-expressing T cells. Immunol. Rev. 257: 107-126.

19. Guedan, S., H. Calderon, A. D. Posey, and M. V. Maus. 2019. Engineering and Design of Chimeric Antigen Receptors. Mol. Ther.—Methods Clin. Dev. 12: 145-156.

20. Xu, Y., X. Tao, B. Shen, T. Horng, R. Medzhitov, J. L. Manley, and L. Tong. 2000. Structural basis for signal transduction by the Toll/interleukin-1 receptor domains. Nature 408: 111-5.

21. Whitlow, M., B. A. Bell, S. L. Feng, D. Filpula, K. D. Hardman, S. L. Hubert, M. L. Rollence, J. F. Wood, M. E. Schott, and D. E. Milenic. 1993. An improved linker for single-chain Fv with reduced aggregation and enhanced proteolytic stability. Protein Eng. 6: 989-95.

22. Chen, X., J. L. Zaro, and W.-C. Shen. 2013. Fusion protein linkers: property, design and functionality. Adv. Drug Deliv. Rev. 65: 1357-69.

23. Reddy Chichili, V. P., V. Kumar, and J. Sivaraman. 2013. Linkers in the structural biology of protein-protein interactions. Protein Sci. 22: 153-67.

24. Matuskova, M., and E. Durinikov. 2016. Retroviral Vectors in Gene Therapy. In Advances in Molecular Retrovirology InTech, Ed. S. K. Saxena.

25. Abken, H. 2017. Driving CARs on the Highway to Solid Cancer: Some Considerations on the Adoptive Therapy with CAR T Cells. Hum. Gene Ther. 28: 1047-1060.

26. Groux, H., A. O'Garra, M. Bigler, M. Rouleau, S. Antonenko, J. E. De Vries, and M. G. Roncarolo. 1997. A CD4+ T-cell subset inhibits antigen-specific T-cell responses and prevents colitis. Nature 389: 737-742.

27. Roncarolo, M. G., S. Gregori, R. Bacchetta, and M. Battaglia. 2014. Tr1 cells and the counter-regulation of immunity: Natural mechanisms and therapeutic applications. Curr. Top. Microbiol. Immunol. 380: 39-68.

28. Gagliani, N., C. F. Magnani, S. Huber, M. E. Gianolini, M. Pala, P. Licona-Limon, B. Guo, D. R. Herbert, A. Bulfone, F. Trentini, C. Di Serio, R. Bacchetta, M. Andreani, L. Brockmann, S. Gregori, R. A. Flavell, and M.-G. Roncarolo. 2013. Coexpression of CD49b and LAG-3 identifies human and mouse T regulatory type 1 cells. Nat. Med. 19: 739-746.

29. Miller, A. D., M. F. Law, and I. M. Verma. 1985. Generation of helper-free amphotropic retroviruses that transduce a dominant-acting, methotrexate-resistant dihydrofolate reductase gene. Mol. Cell. Biol. 5: 431-7.

30. Miller, A. D., and C. Buttimore. 1986. Redesign of retrovirus packaging cell lines to avoid recombination leading to helper virus production. Mol. Cell. Biol. 6: 2895-902.

31. Danos, O., and R. C. Mulligan. 1988. Safe and Efficient Generation of Recombinant Retroviruses with Amphotropic and Ecotropic Host Ranges. Proc. Natl. Acad. Sci. U.S.A. 85: 6460-6464.

32. Bregni, M., M. Magni, S. Siena, M. Di Nicola, G. Bonadonna, and A. Gianni. 1992. Human peripheral blood hematopoietic progenitors are optimal targets of retroviral-mediated gene transfer. Blood 80: 1418-1422.

33. Xu, L., S. K. Stahl, H. P. Dave, R. Schiffmann, P. H. Correll, S. Kessler, and S. Karlsson. 1994. Correction of the enzyme deficiency in hematopoietic cells of Gaucher patients using a clinically acceptable retroviral supernatant transduction protocol. Exp. Hematol. 22: 223-30.

34. Hughes, P. F., J. D. Thacker, D. Hogge, H. J. Sutherland, T. E. Thomas, P. M. Lansdorp, C. J. Eaves, and R. K. Humphries. 1992. Retroviral gene transfer to primitive normal and leukemic hematopoietic cells using clinically applicable procedures. J. Clin. Invest. 89: 1817-24.

35. Sambrook J; Fritsch E F; Maniatis T. 1989. Molecular cloning: a laboratory manual,. Cold Spring Harbor, N.Y.: Cold Spring Harbor Laboratory.

36. 1994. Current Protocols in Molecular Biology, volumes I-III, (R. M. Ausubel, ed). John Wiley & Sons, Inc.

37. 1994. Cell Biology: A Laboratory Handbook, volumes 1-3, (Celis J E, ed). Academic Press.

38. 1994. Current Protocols in Immunology, volumes I-III, (J. E. Coligan, ed). John Wiley & Sons, Inc.

39. Gait J M. 1984. Oligonucleotide Synthesis: a Practical Approach,. Oxford [Oxfordshire]; Washington, D.C.: IRL Press.

40. 1985. Nucleic Acid Hybridisation: A Practical Approach, (B. D. Hames, and S. J. Higgins, eds). Oxford; Washington, D.C.: IRL Press.

41. Hames, B. D., and S. J. Higgins. 1984. Transcription and Translation: a Practical Approach,. Oxford; Washington, D.C.: IRL Press.

42. Freshney, I. R. 1986. Animal Cell Culture: A Practical Approach,. Oxford University Press.

43. 1985. Immobilized Cells and Enzymes: A Practical Approach, (J. Woodward, ed). IRL Press.

44. Perbal B. 1984. Practical Guide to Molecular Cloning,. John Wiley & Sons Inc.

45. 1991. Current Protocols in Immunology, (J. E. Coligan, A. M. Kruisbeek, D. H. Margulies, E. M. Shevach, and W. Strober, eds). John Wiley & Sons Inc.

46. Gross, G., and Z. Eshhar. 1992. Endowing T cells with antibody specificity using chimeric T cell receptors. Faseb J 6: 3370-8.

47. Eshhar, Z., T. Waks, G. Gross, and D. G. Schindler. 1993. Specific activation and targeting of cytotoxic lymphocytes through chimeric single chains consisting of antibody-binding domains and the gamma or zeta subunits of the immunoglobulin and T-cell receptors. Proc Natl Acad Sci USA 90: 720-4.

48. Sadelain, M., R. Brentjens, and I. Rivière. 2013. The basic principles of chimeric antigen receptor design. Cancer Discov. 3: 388-98.

49. Sadelain, M., I. Rivière, and S. Riddell. 2017. Therapeutic T cell engineering. Nature 545: 423-431.

50. June, C. H., M. V Maus, G. Plesa, L. a Johnson, Y. Zhao, B. L. Levine, S. a Grupp, and D. L. Porter. 2014. Engineered T cells for cancer therapy. Cancer Immunol. Immunother.

51. Weinstein-Marom, H., A. Pato, N. Levin, K. Susid, O. Itzhaki, M. J. Besser, T. Peretz, A. Margalit, M. Lotem, and G. Gross. 2016. Membrane-attached Cytokines Expressed by mRNA Electroporation Act as Potent T-Cell Adjuvants. J. Immunother. 39: 60-70.

52. Groux, H., A. O'Garra, M. Bigler, M. Rouleau, S. Antonenko, J. E. De Vries, and M. G. Roncarolo. 1997. A CD4+ T-cell subset inhibits antigen-specific T-cell responses and prevents colitis. Nature 389: 737-742.

53. Gagliani, N., T. Jofra, A. Stabilini, A. Valle, M. Atkinson, M.-G. Roncarolo, and M. Battaglia. 2010. Antigen-specific dependence of Trl-cell therapy in preclinical models of islet transplant. Diabetes 59: 433-9.

54. Lewis, M. D., E. de Leenheer, S. Fishman, L. K. Siew, G. Gross, and F. S. Wong. 2015. A reproducible method for the expansion of mouse CD8+ T lymphocytes. J. Immunol. Methods 417: 134-138.

55. Wirtz, S., and M. F. Neurath. 2007. Mouse models of inflammatory bowel disease. Adv Drug Deliv Rev 59: 1073-1083.

56. Elinav, E., T. Waks, and Z. Eshhar. 2008. Redirection of regulatory T cells with predetermined specificity for the treatment of experimental colitis in mice. Gastroenterology 134: 2014-2024.

57. Elinav, E., N. Adam, T. Waks, and Z. Eshhar. 2009. Amelioration of colitis by genetically engineered murine regulatory T cells redirected by antigen-specific chimeric receptor. Gastroenterology 136: 1721-1731.

1. A nucleic acid molecule comprising a nucleotide sequence encoding an activating chimeric antigen receptor (aCAR) comprising:
(i) an extracellular binding-domain specifically binding an antigen selected from an antigen of the commensal gut microflora and a self-cell surface antigen specific to the lamina propria (LP) or submucosa of the gastrointestinal tract;
(ii) a transmembrane domain;
(iii) an intracellular domain including at least one signal transduction element that activates and/or co-stimulates a T cell; and optionally
(iv) a stalk region linking the extracellular domain and the transmembrane domain.

2. The nucleic acid molecule of claim 1, further comprising a nucleotide sequence encoding a homodimeric IL-10 that is linked to a transmembrane-intracellular stretch, optionally through a flexible hinge.

3. The nucleic acid molecule of claim 1 or 2, wherein the antigen is a toll-like receptor (TLR)-ligand antigen of the commensal gut microflora.

4. The nucleic acid molecule of claim 3, wherein said TLR-ligand antigen is selected from a ligand of TLR1, TLR2, TLR4, TLR5, TLR6, TLR9 and TLR10.

5. The nucleic acid molecule of claim 4, wherein said TLR-ligand antigen is selected from peptidoglycan; a lipopeptide, such as a triacyl lipopeptide; lipoteichoic acid; lipopolysaccharide;
flagellin; bacterial CpG-containing DNA and viral CpG-containing DNA.

6. The nucleic acid molecule of claim 4 or 5, wherein said extracellular binding-domain is selected from the extracellular domain of TLR1, TLR2, TLR4, TLR5, TLR6, TLR9 or TLR10; and a single chain variable fragment (scFv) specifically binding said TLR-ligand antigen.

7. The nucleic acid molecule of claims 6, wherein said extracellular binding-domain is an scFv specifically binding peptidoglycan.

8. The nucleic acid molecule of any one of claims 1 to 7, wherein said intracellular domain comprises at least one domain which is homologous to an immunoreceptor tyrosine-based activation motif (ITAM) of for example, CD3ζ, CD3η chain, or FcRγ chains; to a Toll/IL-1 receptor domain (TIR) of for example TLR1, TLR2, TLR4, TLR5, TLR6, TLR9 or TLR10; or to a co-stimulatory signal transduction element of for example, B cell receptor polypeptide, CD27, CD28, CD278 (ICOS), CD137 (4-1BB), CD134 (OX40), Dap10, CD2, CD5, ICAM-1, LFA-1, Lck, TNFR-I, TNFRII, Fas, CD30; or combinations thereof.

9. The nucleic acid molecule of claim 8, wherein said intracellular domain comprises a tandem arrangement of signal transduction elements selected from TIR-CD28-FcRγ, wherein the TIR is derived from TLR1, TLR2, TLR4, TLR5, TLR6, TLR9 or TLR10; and signal transduction elements of CD28-FcRγ.

10. The nucleic acid molecule of any one of claims 1 to 9, wherein said transmembrane domain is selected from a transmembrane region of a Type I transmembrane protein, an artificial hydrophobic sequence, the transmembrane domain of CD28, CD3ζ, TLR1, TLR2, TLR4, TLR5, TLR6, TLR9 or TLR10, and Fc receptor.

11. The nucleic acid molecule of any one of claims 1 to 10, wherein the aCAR comprises a stalk region linking the extracellular domain and the transmembrane domain, and said stalk region is selected from the stalk of CD28, CD8α, CD8β and the heavy chain of IgG or IgD.

12. The nucleic acid molecule of claim 1 or 2, wherein said antigen is a toll-like receptor (TLR)-ligand antigen of the commensal gut microflora; said intracellular domain comprises at least one domain which is homologous to an immunoreceptor tyrosine-based activation motif (ITAM) of for example, CD3ζ, CD3η chain, or FcRγ chains; to a Toll/IL-1 receptor domain (TIR) of for example TLR1, TLR2, TLR4, TLR5, TLR6, TLR9 or TLR10; or to a co-stimulatory signal transduction element of for example, B cell receptor polypeptide, CD27, CD28, CD278 (ICOS), CD137 (4-1BB), CD134 (OX40), Dap10, CD2, CD5, ICAM-1, LFA-1, Lck, TNFR-I, TNFRII, Fas, CD30, or combinations thereof; said transmembrane domain is selected from a transmembrane region of a Type I transmembrane protein, an artificial hydrophobic sequence, the transmembrane domain of CD28, CD3ζ, TLR1, TLR2, TLR4, TLR5, TLR6, TLR9 or TLR10, and Fc receptor; and the aCAR comprises a stalk region linking the extracellular domain and the transmembrane domain, and said stalk region is selected from the stalk of CD28, CD8α, CD8β and the heavy chain of IgG or IgD.

13. The nucleic acid molecule of claims 12, wherein said TLR-ligand antigen is selected from a ligand of TLR1, TLR2, TLR4, TLR5, TLR6, TLR9 and TLR10; and said intracellular domain comprises a tandem arrangement of signal transduction elements selected from TIR-CD28-FcRγ, wherein the TIR is derived from TLR1, TLR2, TLR4, TLR5, TLR6, TLR9 or TLR10; and signal transduction elements of CD28-FcRγ.

14. The nucleic acid molecule of claims 13, wherein said TLR-ligand antigen is selected from peptidoglycan; a lipopeptide, such as a triacyl lipopeptide; lipoteichoic acid; lipopolysaccharide; flagellin; bacterial CpG-containing DNA and viral CpG-containing DNA.

15. The nucleic acid molecule of claim 14, wherein said extracellular binding-domain is selected from an extracellular domain of TLR1, TLR2, TLR4, TLR5, TLR6, TLR9 or TLR10; and an scFv specifically binding said TLR-ligand antigen.

16. The nucleic acid molecule of claims 15, wherein said scFv specifically binds peptidoglycan.

17. The nucleic acid molecule of claim 1, wherein said aCAR comprises an scFv specifically binding peptidoglycan, a stalk region comprising the hinge of CD8α, a transmembrane domain comprising the transmembrane domain of CD28, and an intracellular domain comprising a tandem arrangement of signal transduction elements of CD28-FcRγ.

18. The nucleic acid molecule of claim 1, wherein said aCAR comprises a TLR, such as TLR2, and the intracellular domain comprises a tandem arrangement of signal transduction elements of CD28-FcRγ linked to the TIR domain of said TLR; or the signal transduction element of CD3ζ.

19. The nucleic acid molecule of claim 2, wherein said homodimeric IL-10 comprises a first and a second IL-10 monomer connected in a single-chain configuration such that the C-terminus of the first IL-10 monomer is linked to the N-terminus of the second IL-10 monomer via a first flexible linker.

20. The nucleic acid molecule of claim 19, wherein said first flexible linker has the amino acid sequence GSTSGSGKPGSGEGSTKG [SEQ ID NO: 5].

21. The nucleic acid molecule of claim 2, wherein said homodimeric IL-10 is linked to the transmembrane-intracellular stretch via a flexible hinge, and said flexible hinge comprises a polypeptide selected from a hinge region of CD8α, a hinge region of a heavy chain of IgG, a hinge region of a heavy chain of IgD; an extracellular stretch of an IL-10R β chain; and a second flexible linker comprising an amino acid spacer of up to 28 amino acids, such as a 21 amino acid spacer consisting of one Gly4Ser(Gly3Ser)2 sequence [SEQ ID NO: 36] and an additional 8 amino acid bridge of the sequence SSQPTIPI [SEQ ID NO: 40].

22. The nucleic acid molecule of claim 21, wherein said transmembrane-intracellular stretch is derived from the heavy chain of a human MHC class I molecule selected from an HLA-A, HLA-B or HLA-C molecule, preferably HLA-A2; or the IL-10R β chain.

23. The nucleic acid molecule of claim 22, wherein the homodimeric IL-10 is linked to the N-terminus of the essentially complete IL-10R β chain.

24. The nucleic acid molecule of any one of claims 2 to 23, wherein said homodimeric IL-10 comprises a first and a second IL-10 monomer connected in a single-chain configuration such that the C-terminus of the first IL-10 monomer is linked to the N-terminus of the second IL-10 monomer via a first flexible linker; said homodimeric IL-10 is linked to the transmembrane-intracellular stretch via a flexible hinge, and said flexible hinge comprises a polypeptide selected from a hinge region of CD8α, a hinge region of a heavy chain of IgG, a hinge region of a heavy chain of IgD; an extracellular stretch of an IL-10R β chain; and a second flexible linker comprising an amino acid spacer of up to 28 amino acids, such as a 21 amino acid spacer consisting of one Gly4Ser(Gly3Ser)2 sequence [SEQ ID NO: 36] and an additional 8 amino acid bridge of the sequence SSQPTIPI [SEQ ID NO: 40]; and said transmembrane-intracellular stretch of said homodimeric IL-10 is derived from the heavy chain of a human MHC class I molecule selected from an HLA-A, HLA-B or HLA-C molecule, preferably HLA-A2; or the IL-10R β chain.

25. The nucleic acid molecule of claim 24, wherein said first flexible linker has the amino acid sequence GSTSGSGKPGSGEGSTKG [SEQ ID NO: 5].

26. The nucleic acid molecule of claim 25, wherein the homodimeric IL-10 is linked to the N-terminus of the essentially complete IL-10R β chain.

27. A composition comprising the nucleic acid molecule of any one of claims 1 to 26.

28. A vector, such as a viral vector, comprising the nucleic acid molecule of any one of claims 1 to 26.

29. A composition comprising at least one vector, wherein the composition comprises one vector of claim 28; or said composition comprises at least two vectors, wherein one vector comprises the nucleic acid molecule of claim 1 and another vector comprises a nucleic acid molecule comprising a nucleotide sequence encoding a homodimeric IL-10 linked to a transmembrane-intracellular stretch, optionally through a flexible hinge.

30. A mammalian regulatory T cell (Treg) comprising the nucleic acid molecule of any one of claims 1 to 26, the vector of claim 28; or a combination of the vector comprising the nucleic acid molecule of claim 1, and a vector comprising a nucleic acid molecule comprising a nucleotide sequence encoding a homodimeric IL-10 linked to a transmembrane-intracellular stretch, optionally through a flexible hinge.

31. The mammalian Treg of claim 30, expressing on its surface an activating chimeric antigen receptor (aCAR) encoded by said nucleic acid molecule.

32. The mammalian Treg of claim 31, having a stable Tr1 phenotype exhibiting the cell-surface markers CD49b and LAG-3.

33. The mammalian Treg of any one of claims 30 to 32, which is a human Treg.

34. A method of preparing allogeneic or autologous Tregs, the method comprising contacting CD4 T cells with the nucleic acid molecule of claim 1 or 2 or a vector comprising it; or a combination of the vector comprising the nucleic acid molecule of claim 1, and a vector comprising a nucleic acid molecule comprising a nucleotide sequence encoding a homodimeric IL-10 linked to a transmembrane-intracellular stretch, optionally through a flexible hinge, thereby preparing allogeneic or autologous Tregs.

35. A mammalian Treg of any one of claims 30 to 32, for use in treating or preventing a disease, disorder or condition in a subject, wherein said disease, disorder or condition is manifested in excessive activity of the immune system, such as an autoimmune disease, allergy, asthma, and organ and bone marrow transplantation.

36. The mammalian Treg for use of claim 35, wherein the autoimmune disease is selected from an inflammatory bowel disease, such as Crohn's disease and ulcerative colitis; celiac disease; type 1 diabetes; rheumatoid arthritis; systemic lupus erythematosus; Sjögren's syndrome; Behçet's disease; scleroderma; collagen vascular diseases; systemic vasculitides, Wegener granulomatosis; Churg-Strauss syndrome; psoriasis; psoriatic arthritis; multiple sclerosis; Addison's disease; Graves' disease; Hashimoto's thyroiditis; myasthenia gravis; vasculitis; pernicious anemia; and atherosclerosis.

37. The mammalian Treg for use of claim 36, wherein the autoimmune disease is selected from an inflammatory bowel disease, such as Crohn's disease and ulcerative colitis; type 1 diabetes; and celiac disease.

38. The mammalian Treg for use of claim 37, wherein the autoimmune disease is an inflammatory bowel disease.

39. The mammalian Treg for use of any one of claims 35 to 38, wherein said subject is human and said mammalian Treg is human.

40. The mammalian Treg for use of claim 39, wherein said Treg is an allogeneic Treg.
